(19) United States
(12) Patent Application Publication
(10) Pub. No.: US 2019/0328670 A1
(43) Pub. Date: Oct. 31, 2019

(54) Microparticles for Oral Delivery

(71) Applicant: Intervet Inc., Madison, NJ (US)

(72) Inventor: Mordechai Harel, Pikesville, MD (US)

(73) Assignee: Intervet Inc., Madison, NJ (US)

(21) Appl. No.: 16/448,539

(22) Filed: Jun. 21, 2019

Related U.S. Application Data
(63) Continuation of application No. 11/597,826, filed on Aug. 23, 2007.
(60) Provisional application No. 60/575,542, filed on May 27, 2004.
(60) Provisional application No. 60/652,893, filed on Feb. 15, 2005.

(51) Int. Cl. A61K 9/19; A61K 9/127; A61K 9/16

(52) U.S. Cl. CPC A61K 9/19; A61K 9/167; A61K 9/127

(57) ABSTRACT

The invention relates to particles suitable for oral administration of a composition to an animal. The particles comprise a substantially indigestible polymer matrix. In one embodiment, the matrix contains the composition and a lipid that is dissoluble in the animal. In another embodiment, the matrix is mixed with the composition and contained in a dissoluble coating. The lipid and coating can be ones which are preferentially dissoluble in one compartment of an animal than in another. For example, the particles can be made to preferentially release the composition in the intestines, rather than the stomach, of a mammal, a fish, or a crustacean. Examples of compounds which can be administered in such compositions include vitamins, fatty acids, oils, carotenoids, nutraceuticals, pharmaceuticals, live or dormant probiotic bacteria, hormones, nucleic acids, and proteins. The particles described herein have the further advantage that they can protect sensitive compounds from oxidation, taste or odor change, and other types of degradation.

### BACKGROUND OF THE INVENTION

The present invention relates to particulate compositions for containing one or more bioactive or other compounds.

The most frequent method of formulating bioactive compounds for oral delivery is microencapsulation. This is usually achieved by the coacervation of the bioactive with one or more digestible polymers, such as gum Arabic, maltodextrin, and gelatin (Chan et al., 2000, J. Microencapsul. 17(6):757-776; Thimma et al., 2003, J. Microencapsul. 20(2):203-210). These applications are realized, in most cases, by the method of atomizing, spraying, or “spray drying”. These techniques are limited in their total loading capacity for the bioactive agents (Madan et al., 1972, J. Pharm. Sci. 61:1586-1593; Chan et al., 2000, J. Microencapsul. 17(6):757-776; Hamdi et al., 2001, J. Microencapsul. 18(3):373-383).

Soluble starch containing a high concentration of amylopectin polymer is used in numerous applications in the food industry, for example as a swelling agent and for accelerated and extended water absorption in foods such as soups, sauces, instant puddings, baby food, and thickening agents. However, the use of starch as the sole matrix material generally results in a matrix that releases the encapsulated material quickly. Penetration of water into a pure starch matrix causes early release of the encapsulated product into the environment. Generally the release time of the encapsulated product is too short to provide a time-release or controlled-release effective for delivering the encapsulated product to a desired location or time.

A shortcoming of existing encapsulation techniques and materials is that they do not protect odor and taste of encapsulated oily products or provide significant gastric protection. Pre-emulsification of the oils or heating steps in existing encapsulation methods can cause oxidation and/or rapid degradation leaving the oil or oil-associated bioactive compound(s) susceptible to digestion.

A common problem associated with the oral application of functional foods and drugs is the loss of activity by oxidation, chemical decomposition during storage, preparation, or in the animal's digestive system before absorption. The harsh environment of some food processes, like milling, mixing, and extrusion, destroys a significant portion of bioactive materials before they become finished food products. This is especially true for live probiotic bacteria. Most types of conventional food processing are designed for complete or partial sterilization of the food product to eliminate or reduce bacterial contamination (including beneficial probiotic bacteria). Food scientists and application specialists are continuously searching for methods to protect bioactive compounds, including probiotic bacteria, against decomposition during processing and storage.

Additional problems result from the interaction between the desired bioactive compounds and other ingredients, such as metal chelators, surfactants, and hygroscopic ingredients. Examples of problems associated with such interactions include sensitivity of probiotic bacteria to surfactants (such as lecithin and TWEEN® 80), which are added to or inherently found in some foods, and the sensitivity of unsaturated fatty acids found in certain omega-3 rich oils to certain metal ions typically added to feeds, such as iron (Capra et al., 2004, Lett. Appl. Microbiol. 38: 499-504; Margolles et al., 2003, Int. J. Food Microbiol. 82: 191-198; Frankel et al., 2002, J. Agric. Food. Chem. 50: 2094-2099).

One known way to retain activity and effect appropriate release of a bioactive agent is encapsulation. It is known to provide solid particulate materials in which a bioactive agent is contained and protected in a particulate matrix. Various attempts have been made to embed bioactive agents in many different types of organic matrices, including proteins, carbohydrates, and solid fats among others. The aim of encapsulation is to provide stable free-flowing powders that contain the encapsulated bioactive agent in a form easily incorporated into foods and other products.

Most encapsulation methods produce water-soluble particles. A number of water-soluble carrier materials are employed in production of this type of encapsulation, such as proteins, sugars, modified starches, and gums (e.g., see International Patent Application Publication no. WO 2004/082660). The encapsulated materials are generally produced by spray drying, extrusion, or fluidized bed coating. However, these types of encapsulation are not suitable for protecting bioactive agents in food products that contain water or have a high water activity because of dissolution and subsequent degradation of the encapsulated bioactive materials upon contact with the food product. Since water is involved at one or more stages of processing and storage operations for most foods, encapsulation in water-soluble matrices has limited applicability for improving the stability the of bioactive compound or for controlling retention and directed release of bioactive agents.

To overcome the problem of degradation of the microcapsule matrix during processing or storage in humid environments, or for the production of a food or feed with a high water activity, others have employed fat encapsulation or top-coating of water-soluble particles with a protective layer of wax. Examples of such methods include those disclosed in U.S. Pat. Nos. 4,350,679, 5,789,014, and 5,258,132. Use of fat coating is limited to food products that are processed at temperatures below the melting point of the fat. This process is not applicable for a typical food process that includes boiling, baking, spray drying, or extruding because the coating fat can become liquefied and its protective properties can be lost.

Another known encapsulation method is microencapsulation by coacervation. The encapsulation of bioactive agents into coacervated microcapsules is described, for example in International Patent Application Publication nos. WO 93/19621 and WO 93/19622. Microencapsulation by coacervation creates a barrier of protein around a droplet of functional oil, such as an essential oil or a mixture of omega-3 fatty acids (e.g., docosahexaenoic, arachidonic, or eicosapentaenoic acids). This barrier improves retention during heat processing and increases shelf-life stability. The protein surrounding the oil is mostly soluble and is broken down by the proteases and acid pH of the stomach thereby releasing the oil (or other bioactive agents) into the harsh environment of the stomach. Coacervated microcapsules can be easily ruptured during conventional food manufacturing processes as a consequence of the shear forces applied during mixing, grinding, or other high-shear processes to which the product is subjected during its production.

Others have prepared microparticles using polysaccharide materials, such as alginate, pectin, and gellan gums. Alginate, in particular, has found useful application as a water insoluble matrix for the encapsulation of cells, drugs, vitamins and colorings (see, e.g., U.S. Pat. Nos. 4,389,419 and 4,3627,48). However, for encapsulation of oxidation- and humidity-sensitive bioactive compounds, alginate and other heat-stable polysaccharides exhibit poor barrier properties. Furthermore, the relatively large pore sizes of these polysaccharides restrict the capability of alginate beads to act as an insoluble barrier for small molecules, such as small peptide hormones, drugs, flavor molecules, free amino acids, or vitamins. Bioactives of high volatility and water-solubility simply cannot be encapsulated and retained in such a matrix.

In view of the shortcomings in known methods of encapsulating bioactive agents and other compounds, it would be advantageous to have an alternative method of encapsulating that permits incorporation of a significant amount of the desired ingredient into a microparticle. The particle should preferably exhibit high stability in high water activity environments, a high degree of resistance to gastric conditions, and good release kinetics for the encapsulated ingredient, for example to the absorptive or otherwise appropriate regions of the intestine. The present invention overcomes the shortcomings of the prior art and provides such particles and methods of making and using them.

### BRIEF SUMMARY OF THE INVENTION

The invention relates to particles for orally administering a composition to an animal.

In one embodiment, the invention relates to a particle that includes a substantially indigestible polymer matrix. Suspended in the matrix are the composition and a lipid that is dissoluble in the animal. The lipid and the composition can be admixed, emulsified, or otherwise combined. The matrix can, for example, be made from one or more polysaccharides, proteins, synthetic polymers, or some combination of these.

In another embodiment, the invention relates to a particle that includes a mixture (e.g. a dispersion or emulsion) of a substantially indigestible polymer matrix and an oily composition. This mixture is contained within a coating that is dissoluble in the animal. Beneficially, the particle can also include an emulsifier and/or water. The coating can, for example, be one or more of a cross-linked polysaccharide, a protein, or some other dissoluble material.

The particles described herein can be used to deliver bioactive agents (e.g., nutrients, drugs, vaccines, antibodies, and the like), bacteria (e.g., probiotic bacteria), smaller particles, or substantially any other material to the animal.

The invention also includes methods of making the particles described herein and methods of using the particles to deliver compositions to animals.

### BRIEF SUMMARY OF THE SEVERAL VIEWS OF THE DRAWINGS

The patent or application file contains at least one drawing executed in color. Copies of this patent or patent application publication with color drawing(s) will be provided by the Office upon request and payment of the necessary fee.

FIG. 1 is an image of dry high-amylose starch granules under light microscope at 100× magnification.

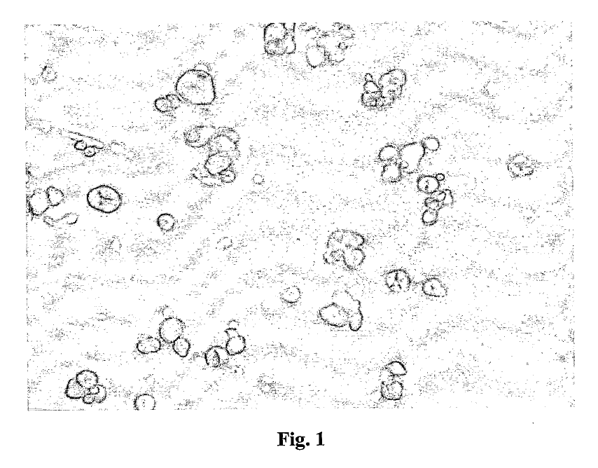

FIG. 2 is an image of swollen high-amylose starch granules in aqueous solution after being subjected to mild base and temperature treatment.

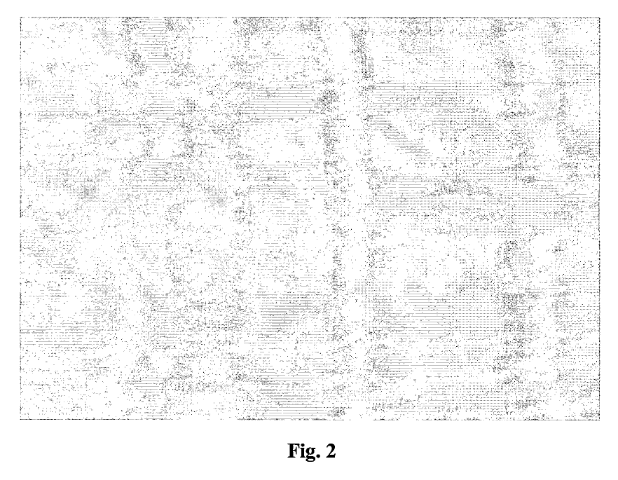

FIG. 3 is an image that depicts breakdown (collapse) of the swollen high-amylose granules and the formation of a non-digestible polymeric complex after the addition of lecithin.

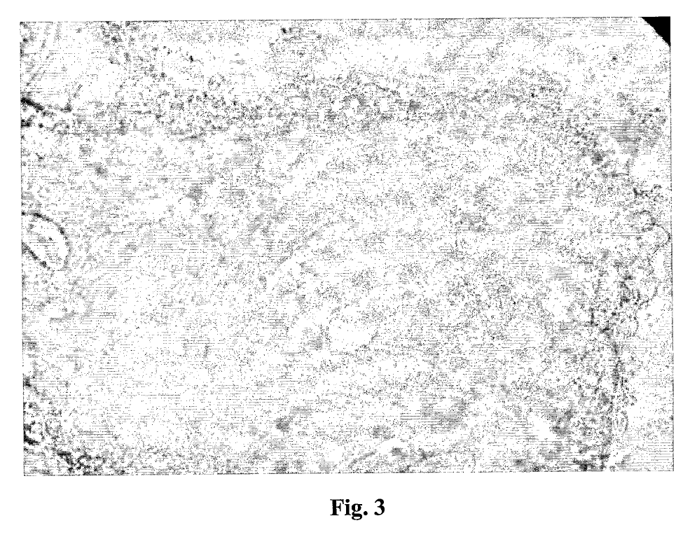

FIG. 4 is an image of wet microbeads comprised of non-digestible polymers embedded in a matrix of alginate.

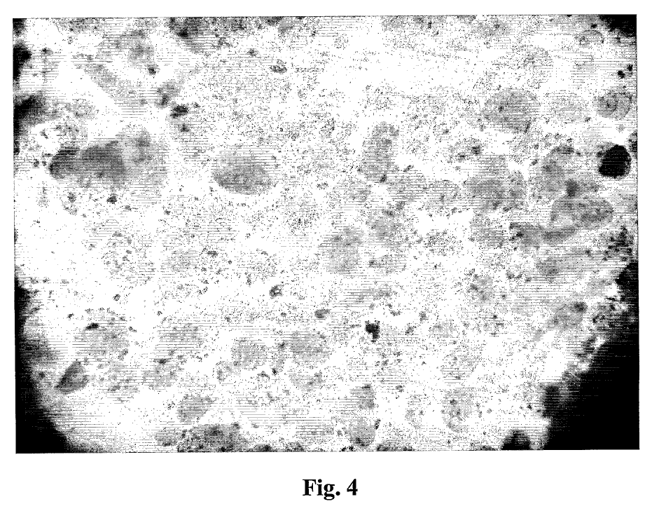

FIG. 5 is a color image of solid fat droplets immobilized in alginate matrix.

FIG. 6 is a diagram of in-line mixing and preparation of solid fat/probiotic/hydrocolloid mixture and spray capture in a chilled tank containing a solution of 1% of calcium chloride.

FIG. 7 is a graph which illustrates stability of two microparticulate preparations of Lactobacillus acidophilus GG over a 30-day storage period at 4 degrees Celsius. Lot PMJ0304A3 is made with liquid oil while PMJ0404A3 is made using cocoa butter.

FIG. 8 is a graph which illustrates stability of two microparticulate preparations of Lactobacillus acidophilus GG maintained at 50 degrees Celsius for up to 2 hours. Lot PMJ0304A3 is made with liquid oil while PMJ0404A3 is made using cocoa butter. The right hand scale is in % survival versus initial counts and corresponds to the dotted lines.

FIG. 9 is a bar graph which illustrates survival after four days of dry Lactobacillus rhamnosus encapsulated in liquid (mineral oil) or solid (fish oil wax) oil-alginate matrix in open air and room temperature environments.

FIG. 10 is a table which lists data reflecting retention of oil droplets in alginate-high amylose starch matrix after exposure in 70 degrees Celsius water and in artificial gastric and intestinal juices.

FIG. 11 is a color photograph of three vials, illustrating solubility of solid oil/astaxanthine droplets embedded in alginate-high amylose starch matrix (vial labeled 4) after exposure in water (vial labeled 1) and in artificial intestinal juice (vial labeled 2). The particles are insoluble in water but are completely dissolved and release the active agent in the lower digestive tract.

### DETAILED DESCRIPTION OF THE INVENTION

The invention relates to particles suitable for orally administering a composition to an animal. Two overlapping types of particles are described here. Although the two types are not mutually exclusive, the types are referred to herein as microparticles and microbeads.

The microparticles have a matrix formed of a substantially indigestible polymer. Suspended (e.g., enclosed or dispersed) in that matrix are the composition to be administered and a lipid that is soluble in the animal. The lipid and the composition are preferably mixed, blended, emulsified, or otherwise mingled. The lipid can be one which is preferentially soluble in one bodily compartment (e.g., the intestines generally, or the large or small intestine) of the animal, relative to another compartment (e.g., the stomach). Furthermore, the lipid can be one which is more dissoluble in one species of animal than in another. The lipid can also enhance the stability or degradation resistance of the composition to be administered. Lipids which are solid or waxy at the normal storage temperature of the microparticles provide a superior humidity barrier for probiotic bacteria and other humidity-sensitive bioactive compounds over an extended period of time, for example.

The microbeads also have an indigestible polymer matrix. That matrix is mixed with the oily composition to be administered to the animal. The mixture is contained within a coating that is dissoluble in at least one compartment of the animal to which the composition is to be administered. Preferably, the matrix and the oily composition are emulsified, in which case an emulsifier is preferably included in the mixture as well. The microbeads can include water, and such water can be part of an emulsion of the matrix and the oily composition.

The particles described herein can be prepared and used as free-flowing dry powders, slurries, suspensions, and the like, and are useful for delivering to an animal a drug, a pesticide, a nutrient, a vaccine, a smaller particle, or substantially any other composition that can be contained in the particles. The particles are thus suitable for use in human food products, animal feeds (e.g., pet foods and farmed animal diets), therapeutic compositions (e.g., drugs), prophylactic compositions (e.g., vaccines, antibiotics, and probiotic bacterial preparations), and pest control products among other products.

The particles described herein unexpectedly provide both a large loading capacity (especially for oils and oil-associated compounds) and exceptional resistance to degradation by gastric enzymes. The particles protect the composition from degradation by oxidation, interaction with humidity or water, or interaction with components (e.g., gastric fluid) of an animal compartment to which delivery of the composition is not desired. Apart from permitting selection of the animal compartment to which the composition is delivered, the barrier properties of the particles allow simplified and prolonged storage of a product that contains them.

Definitions

As used herein, each of the following terms has the meaning associated with it in this section.

A “particle” is a discrete piece of a (homogeneous or heterogeneous) material having a maximum dimension not greater than 5000 micrometers.

A “microbead” is a dry or wet particle that includes at least one substantially indigestible polymer admixed with an oil or with an oil-associated bioactive agent and coated with a soluble coating.

A “microparticle” is a dry or wet particle includes at least one substantially indigestible polymer in which is suspended (either separately or in combination) a composition to be administered to at least one compartment of an animal and a lipid that is soluble in that compartment.

An “encapsulate” is any compound that is enclosed, suspended, or contained within the confines of a microparticle or a microbead.

An “encapsulant” is a matrix material or coating material used to form the matrix in which an encapsulate is constrained. An encapsulant can act both as a coating and as a matrix material.

A matrix is “substantially indigestible” by an animal if the matrix does not substantially lose its structural cohesion in the stomach of the animal during the normal period of gastric residence following oral administration of the matrix to the animal. It is recognized that gastric residence time can differ based on fasting state, gastric contents, particle size, and other factors. A skilled artisan understands that these factors and the chemical identity of a matrix can be made to correspond, for example by modulating fasting near the time of oral administration of the matrix.

A matrix is “digestible” by an animal if the matrix substantially loses its structural cohesion in the stomach of the animal during the normal period of gastric residence following oral administration of the matrix to the animal.

“Gelatinization” of starch refers to reduction in hydrogen bonding between amylopectin and amylose which are responsible for the integrity of starch granules. When an aqueous suspension of starch is heated to a certain temperature, the hydrogen bonding weakens and the starch granule swells until collapsing.

A “bioactive agents” broadly refers to any composition that produces a desired result upon administration to an animal. Examples of bioactive agents are probiotic microorganisms, liposomes, compounds such as proteins, drugs, poisons, vitamins, minerals, imaging contrast agents, colorants, and preservatives.

The terms “drug,” “therapeutic agent,” or “pharmaceutical” include any physiologically or pharmacologically active substance that produces a localized or systemic effect or effects in a human or another animal to which it is administered. By way of example, such substance include compounds known or believed to cure, mitigate, prevent, or attenuate a disease in an animal.

An “animal” is any organism in the taxonomic Kingdom Animalia. By way of example, animals include mammals, fish, birds, reptiles, amphibians, crustaceans, and rotifers. Other examples include domestic household, sport, zoo, or farm animals such as sheep, goats, cattle, horses, pigs, laboratory animals such as mice, rats and guinea pigs, hatchery fish, farmed birds, and fanned reptiles.

An “invertebrate” is an animal that is not in the taxonomic Subphylum vertebrata. Examples of invertebrates include arthropods (including shrimp and insects, for example), acarids, crustacea, mollusks, nematodes, and other worms.

A “bioadhesive” particle is a particle having a surface capable of binding with a biological membrane such that it is retained on that membrane for longer period of time than a particle not capable of forming any covalent or non-covalent attachment with the membrane. Examples of bioadhesive materials that are known in the art include cyclodextrins, enamines, malonates, salicylates, glycyrrhetinates, chitosans, and glucans.

A “permeability enhancing agent” refers to a compound or combination of compounds that increases the ability of a chemical species to pass across a biological membrane. Known examples of such agents include bile salts, deoxycholates, fatty acids, fatty acid salts, acyl glycerols, tyloxapols, acyl carnitines, phospholipids, lysophosphatides, and fusidates.

A composition is “dissoluble” in a compartment of an animal if the composition either dissolves in a liquid present in the compartment or is in a liquid phase under the conditions (e.g., temperature and pH) that occur in the compartment.

Description

The invention relates to particles for administering a composition to an animal, including administration to a selected compartment (e.g., stomach, small intestine, or large intestine) of the animal. The particles are of two overlapping and related types, referred to herein as microparticles and microbeads. For the sake of convenience, these two types of particles are described in separate sections herein, after which are discussed the identities and properties of ingredients useful in one, the other, or both, as well as uses for both types of particles.

Microparticles

The microparticles described herein comprise a matrix that includes at least one substantially indigestible polymer. The composition to be administered to the animal is suspended in the substantially indigestible polymer matrix. A lipid that is dissoluble in the desired compartment of the animal is also suspended in the matrix.

Each of the lipid and the composition can be separately enclosed or dispersed in the matrix. Preferably, however, the lipid and the composition are combined prior to suspension in the matrix. If the composition (e.g., a vitamin) is soluble in the lipid, then the composition can be simply dissolved in the lipid. If the composition and the lipid are immiscible, then they can be separately suspended in the matrix, suspended as immiscible droplets, or suspended in the form of an emulsion. If desired, the matrix, the lipid, and the composition can be formed into a single emulsion in which the matrix is present in the continuous phase. In microparticles that include an emulsified component, an emulsifier can be used to facilitate formation or stability of the emulsion.

The lipid can be one which is preferentially soluble in one bodily compartment (e.g., the intestines generally, or the large or small intestine) of the animal, relative to another compartment (e.g., the stomach). Furthermore, the lipid can be one which is more soluble in one species of animal than in another. The lipid can also enhance the stability or degradation-resistance of the composition to be administered. Lipids which are solid or waxy at the normal storage temperature of the microparticles provide a superior humidity barrier for probiotic bacteria and other humidity-sensitive bioactive compounds over an extended period of time, for example. Such lipids can also substantially impair passage of oxygen to oxidation-sensitive components.

The microparticles can be as large as 5 millimeters along their largest dimension. There is no lower limit on the size of the microparticles, but microparticles having a maximum dimension not smaller than 10 micrometers are preferred. The maximum desired size of a microparticle can also depend on the use to which it is to be put. For example, very small animals (e.g., rotifers) are not able to consume particles greater than the size of their mouths. Furthermore, when the microparticles are to be used as components of a food product, it can be desirable that the microparticles are not visible. In each of these instances, appropriate sizes for particles would be apparent to a skilled artisan in the corresponding field.

In one embodiment, a composition including a bioactive substance (e.g., a nutraceutical, pharmaceutical, vaccine, probiotic, hormone, vitamin, or protein ingredient) is embedded in a solid or waxy lipid matrix. The matrix is immobilized in an substantially indigestible polymer matrix to form a microparticle that releases the bioactive compound in the digestive tract of an animal. Such a microparticle is useful for protecting bioactive materials from humidity and oxidation damage during storage, for decreasing degradation of the bioactive material contained therein by gastric conditions, and for limiting release of the bioactive material from the microparticle when it contacts water below the melting point of the solid lipid. Microparticles are also useful for improved the heat and shear stability of the a composition contained therein during storage and processing (e.g., during preparation of food products into which the microparticles are incorporated).

In one embodiment, the invention relates to a method of preparing a composition containing unsaturated oils, such as fish oil, in a complex effective to stabilize the unsaturated oils. The composition comprises the oil mixed with a lipid that is solid or waxy at the temperature at which the microparticles are normally stored, but which is liquid at the body temperature of an animal. The mixture can be embedded in, suspended in, or mixed with a substantially indigestible (e.g., amylose) matrix. Upon delivery to the stomach or intestine of an animal, the lipid becomes liquid, permitting egress of the oil from the microparticles. Mechanical digestive activity, pH, temperature, enzymatic activity, presence of digestive juices, or some combination of these can further enhance release of the oil from the microparticles. Other (e.g., hydrophilic) bioactive agents can be used in place of the oil and can be similarly delivered.

The shape of the microparticles is not critical, and can be influenced by the manufacturing processed used to make them, the requirements of the use to which they are to be put, aesthetic concerns, or other factors. By way of example, the microparticles can be roughly spherical, cubical, cylindrical, needle-shaped, disc-like, flake-like, or irregularly-shaped. The microparticles used in a particular application need not be uniform in size or shape, or even nearly so. Nonetheless, it is recognized that the more nearly identical microparticles in a preparation are, the more uniform their properties (e.g., dissolution or release rate) will be. A skilled artisan in a corresponding field is able to select microparticles of appropriate size, shape, and uniformity based on the use to which they will be put.

The microparticles can optionally include other components, such as flavoring or coloring agents, preservatives, and one or more coatings, such as a coating that is dissoluble in a compartment of an animal to which the microparticles are to be delivered. The microparticles can be incorporated into other compositions, such as food products, animal feeds, vitamin tablets, and the like.

Microbeads

The microbeads described herein include an indigestible polymer matrix. That matrix is mixed with a composition to be administered to the animal. The microbeads are particularly amenable for administration of oily compositions. Surrounding (entirely or partially) the mixture is a coating that is dissoluble in at least one compartment of the animal to which the composition is to be administered.

The matrix and the oily composition can be, and preferably are, emulsified. Such an emulsion can be formed using only the matrix and the composition, where possible. An emulsifying agent can be added, as can water or one or another solvent. More than one solvent can be used, and each solvent can be miscible with one or both of the matrix and the composition. Alternatively, the oily composition can be dispersed in the matrix.

The matrix having the oily composition embedded, dispersed, or otherwise mixed therein is contained in a coating. Preferably, the coating completely covers the surface of the mixture. However, porous or discontinuous coatings can be used. The identity of the coating is not critical. Coatings that include soluble polymers (e.g., polysaccharides) that can be cross linked using inexpensive reagents (e.g., acids, bases, and metal or divalent cations) are preferred.

Including oily compositions in the microbeads described herein improves the heat and storage stability of the compositions and of any product (e.g., food, pharmaceutical, or animal feed products) that contain the microbeads. The microbeads can also suppress any offensive flavor or odor that may be attributable to the oily composition(s) they contain, thereby improving the flavor or odor of, for example, a food composition containing the composition(s). The microbeads also stabilize the composition(s) against thermal, oxidative, and other chemical degradation.

In one embodiment, the invention relates to a method of preparing a composition containing unsaturated oils, such as fish oil, in a complex effective to stabilize the unsaturated oils. The composition comprises one or more such oils entrapped in microbead particle, such as a particle having a substantially indigestible (e.g., amylose) matrix containing or mixed with the oils. The microbead can include a digestible component to enhance dissolution or water permeation of the microbead or of the matrix. The microbead can have a coating that is dissoluble in, for example, the stomach of an animal. The bioactive compound(s) can be released in the gastrointestinal system of an animal by mechanical digestive activity, pH, temperature, enzymatic activity, or some combination of these.

In another embodiment, the invention includes particles made by combining an oil (which can be or include a bioactive agent), a substantially indigestible polymer matrix, and an emulsifier. This mixture can be emulsified and coated with a dissoluble (e.g., digestible) polymer matrix. Such particles can be orally administered to effect delivery of the oil, the bioactive agent, or both, to the gastrointestinal tract of an animal. In this embodiment, the dissoluble polymer matrix can, for example, be a soluble polysaccharide that forms cross-links in the presence of acid, base, metal or divalent cations.

Others have observed that addition of emulsifiers such as lecithin to an oil can improve the stability of the oil, especially for unsaturated fatty acid-containing oils. This stabilizing effect can be expected to occur in microbeads which include an emulsifier, in addition to the other protective effects described herein. Inclusion of an emulsifier at ratios of from 1 part emulsifier (e.g., lecithin) to 1 to 10 parts oil is effective for stabilization of the oil against oxidation. Inclusion of an emulsifier can be effective for stabilizing various oils, including polyunsaturated fatty acid-(PUFA-)containing oils, for example.

The microbeads described herein are particularly suitable for administration of oily substances to animals. Such substances can include purified or crude oils, and may include substantially any organic or inorganic oil, whether natural or synthetic. The oils may consist of or include triglycerides, such as any of the known vegetable or essential oils. Examples of suitable oils include safflower oil, sunflower oil, canola oil, corn oil, peanut oil, pine oil, lilac oil, fish oil, squid oil, polar oils, non-polar oils, medium chain triglyceride (MCT) oils, jojoba oil, and the like. Suitable oils can contain dispersed, dissolved, or suspended materials.

The microbeads can be as large as 5 millimeters along their largest dimension. There is no lower limit on the size of the microparticles, but microparticles having a maximum dimension not smaller than 5 micrometers are preferred. The maximum desired size of a microbead can also depend on the use to which it is to be put. For example, very small animals (e.g., rotifers) are not able to consume particles greater than the size of their mouths. Furthermore, when the microbeads are to be used as components of a food product, it can be desirable that the microparticles are not visible. In each of these instances, appropriate sizes for particles would be apparent to a skilled artisan in the corresponding field.

The shape of the microbeads is not critical, and can be influenced by the manufacturing processed used to make them, the requirements of the use to which they are to be put, aesthetic concerns, or other factors. By way of example, the microbeads can be roughly spherical, cubical, cylindrical, needle-shaped, disc-like, flake-like, or irregularly-shaped. The microbeads used in a particular application need not be uniform in size or shape, or even nearly so. Nonetheless, it is recognized that the more nearly identical microbeads in a preparation are, the more uniform their properties (e.g., dissolution or release rate) will be. A skilled artisan in a corresponding field is able to select microparticles of appropriate size, shape, and uniformity based on the use to which they will be put.

The microbeads can optionally include other components, such as flavoring or coloring agents, preservatives, and one or more coatings, such as a coating that is dissoluble in a compartment of an animal to which the microparticles are to be delivered. The microparticles can be incorporated into other compositions, such as food products, animal feeds, vitamin tablets, and the like.

Substantially Indigestible Polymer Matrix

The particles (i.e., microparticles and microbeads) described herein include a polymer matrix that is substantially insoluble. The chemical identity of the polymer or polymers used to form this matrix is not critical. The function of the matrix is to provide a relatively cohesive mass capable of securing (e.g., containing, sticking to, or mixing with) the composition to be delivered to the animal. The material from which the matrix is made will depend on the required strength, stability, solubility, and other properties required for the particular application for which the particles are to be used. A skilled artisan in the corresponding field is able to select an appropriate polymer or combination of polymers to achieve such uses.

Many substantially indigestible polymers are known in the art. It is also recognized that the digestibility of a polymer depends on the identity of the animal to which the polymer is to be administered (or, more specifically to the characteristics of the animal's digestive system), the expected residence time of the particle in the digestive system of the animal, and the presence, absence, and characteristics of any coating that may shield the polymer from the digestive system. The degree of digestibility that is acceptable for a polymer will also depend on the amount of polymer digestion that can be tolerated for the particular use. Each of these factors can be used by a skilled artisan to select an appropriately indigestible polymer.

Examples of substantially indigestible polymers that can be used in the particles described herein include polyvinylpyrrolidones, polyvinyl alcohols, polyethylene oxides, celluloses and their derivatives, silicone polymers, polyhydroxyethylmethacrylates, and starches and their derivatives (e.g., high-amylose starch preparations).

The substantially indigestible polymer can, for example, be a precipitatable hydrocolloid, including any carbohydrate that hydrates and forms a gel in a solution and then precipitates by changing the temperature and/or pH of the hydrocolloid solution, or by cross linking with divalent cations or metal ions. Examples of precipitatable hydrocolloids include starch, modified starch and starch derivatives, cellulose, glycogen, inulin, chitin, chitosan, pectin, chondroitin and alginic acid and a gum, such as acacia gum, guar gum, agar, alginates, carrageenan, locust bean gum and xanthans.

A great deal of research has been performed by others in the field of starch chemistry, and methods of making starch preparations having desired properties are relatively well established. In particular, methods of making digestible and substantially indigestible starch preparations are known.

Naturally-occurring starches are composed of two primary fractions, designated amylose (straight-chain starch) and amylopectin (branched-chain starch). Amylose and amylopectin differ not only in their chemical structures but also in their susceptibility to digestion, their stability in dilute aqueous solutions, their gel texture, and their film properties.

Water insoluble starch is high amylose starch having a granular shape similar to the shapes, which occur in native starch. Granular starch can have various shapes and sizes (usually in the range of 0.5-200 micrometers) and is usually semi-crystalline. It is not soluble in cold water without the use of chemicals or heat. Insoluble starch swells to a limited extent only (the water uptake is generally limited to less than 5 times its own weight). Insoluble starch can be chemically or physically modified starch such that most of the original shape and size is maintained after modification. Suitable derivatives are oxidized starch (e.g., carboxy starch, dialdehyde starch), carboxyalkylated starch, sulfated or phosphorylated starch, cationic starch, and the like. The modified granular starches do not form gels in cold water without the addition of chemicals.

Water-insoluble starch will tend to be substantially indigestible, since digestive enzymes are unable to break up crystalline regions of the starch. As a general rule, starches having a high amylose/amylopectin ratios (more than 0.5) will tend to be substantially indigestible. Natural insoluble starch has various granular shapes and sizes (usually in the range of 0.5-200 micrometers), or can be chemically or physically modified wherein most of the original shape and size is maintained after modification. Suitable derivatives are oxidized starch (e.g., carboxy starch, dialdehyde starch), carboxyalkylated starch, sulfated or phosphorylated starch, cationic starch, and the like.

The use of insoluble starches provides advantages over the use of soluble starches. Higher starch concentrations or starches with higher molecular weights can be used. Thus, a polymeric matrix can be prepared that has a high network density, which may be advantageous for tight control of product release properties. Another advantage of using insoluble starch is that various types of starches can be used, such as high-amylose starch, which cannot be digested in the stomach (i.e., non digestible; Lenaerts et al., 1998, J. Control. Release 53(1-3):225-234; Champ et al., 1998, Am. J. Clin. Nutr. 68(3):705-710; Asp et al., 1987, Scand. J. Gastroenterol. Suppl. 129:29-32). The proportion of amylose and amylopectin polymers in the starch allows for adjustment of the network structure such that the release properties of the encapsulate can be adjusted. A skilled artisan in this field is able to make such adjustments. For example, release in the gastrointestinal tract may be spread out or delayed (i.e., resulting in release in the intestines, rather than the stomach) as a result of the presence of these substantially in digestible crystalline structures.

By way of example, a non-digestible starch preparation (typically including) can be made by gelatinizing a starch that contains at least 70% amylose in warm water (e.g. 40-60 degrees Celsius) at a high pH (e.g., pH 10-12). An emulsifier, such as egg or soy lecithin, is added to dissolve the swollen starch granules and the pH is reduced to pH 7-8, thereby forming a soluble complex comprising a non-digestible starch matrix.

Bioactive Compositions

The particles described herein can be used to deliver substantially any chemical species, combination of chemicals, cell, or other piece of matter that can be incorporated into the particle to a component of an animal. All such items are referred to herein as “bioactive” compositions, regardless of what the utility of the composition is. Bioactive compositions include, for example, pharmaceutical compositions or compounds, nutraceutical compositions or compounds, nutritional components, probiotic bacteria, bacteriophages, viruses, flavorants, fragrances, detergents or other surface-active compositions.

Examples of these agents include antibiotics, analgesics, vaccines, anti-inflammatory agents, antidepressants, anti-viral agents, anti-tumor agents, enzyme inhibitors, formulations containing zidovudine, proteins or peptides (such as vaccines, antibodies, antimicrobial peptides), enzymes, (e.g., amylases, proteases, lipases, pectinases, cellulases, hemicellulases, pentosanases, xylanases, and phytases), liposomes, aromatic nitro and nitroso compounds and their metabolites, HIV protease inhibitors, viruses, and steroids, hormones or other growth stimulating agents, pesticides, herbicides, germicides, biocides, algicides, rodenticides, fungicides, insecticides, antioxidants, plant and animal growth promoters, plant and animal growth inhibitors, preservatives, nutraceuticals, disinfectants, sterilization agents, catalysts, chemical reactants, fermentation agents, foods, animal feeds, food or animal feed supplements, nutrients, flavors, colors, dyes, cosmetics, drugs, vitamins, sex sterilants, fertility inhibitors, fertility promoters, air purifiers, microorganism attenuators, nucleic acids (e.g., RNA, DNA, PNA, vectors, plasmids, ribozymes, aptamers, dendrimers, and the like), antioxidants, phytochemicals, hormones, vitamins (such as vitamins A, B1, B2, B6, B12; C, D, E, and K, pantothenate, and folic acid), pro-vitamins, carotenoids, minerals (such as calcium, selenium, magnesium salts, available iron, and iron salts), microorganisms (such as bacteria, such as probiotics, lactobacilli, fungi, and yeast), prebiotics, trace elements, essential and/or highly unsaturated fatty acids (such as omega-3 fatty acids, and mid-chain triglycerides), nutritional supplements, enzymes (such as amylases, proteases, lipases, pectinases, cellulases, hemicellulases, pentosanases, xylanases, and phytases), pigments, amino acids, agriculturally useful compositions to either prevent infestation (such as herbicides, pesticides, insecticides, rodenticides, fungicides, mixtures thereof) or to promote growth (such as hormones, fertilizers, or other growth stimulating agents), flavorants, and fragrances.

Oil-associated bioactive compounds and/or other bioactive compounds and microbes are added and mixed thoroughly into the complex solution in a final concentration of between about 0.1% to about 80% by weight of the microbead.

The particles described herein can be used to deliver organism-based bioactive agents including bacteria (e.g., Bacillus spp., B. licheniformis, B. subtilis, Lactobacillus spp., L. bulgaricus, L. helviticus, L. plantarum, L. paracasei, L. casei, L. rhamnosus, L. lactis, Alteromonas spp., A. media, Carnobacterium spp., C. divergens, Vibrio spp., V. alginolyticus, Pseudomonas spp., P. fluorescens, Streptococcus spp., S. lactis, S. thermophilus, Pseudoalteromonas spp., P. undina), yeast (e.g., Saccharomyces spp., S. cerevisiae, S. exiguous, Phaffia spp., P. rhodozoma, Pichia spp., P. pastoris, Kluyveromyces spp., K. aestuarii, K. marxianus, and K. yarrowii., Schizochitrium, Ulkenia, Crypthecodinium, Nannochloropsis, nannochloris, Hematococcus, Pfaffia, Isochrysis and Chlorella) and viruses (e.g., live viruses, heat killed viruses, attenuated viruses, bacteriophages).

The particles described herein can also be used to deliver antimicrobial-based bioactive agents including, but not limited to gentamicin, tetracycline, oxytetracycline, doxycycline, ampicillin, ticarcillin, cephalothin, cephaloridine, cefotiam, cefsulodin, cefmenoxime, cefmetazole, cefazolin, cefotaxime, cefoperazone, ceftizoxime, moxolactam, latamoxef, thienamycin, sulfazecin, and azthreonam.

The particles described herein can be used to deliver hormone-based bioactive proteins including, but not limited to somatostatin, somatostatin derivatives, growth hormones, prolactin, adrenocorticotropic hormone, melanocyte stimulating hormone, thyroid hormone releasing hormone (TRH), TRH salts, TRH derivatives, thyroid stimulating hormone, leutinizing hormone, oxytocin, calcitonin, gastrin, secretin, pancreaozymin, choecystokinin, interleukins, thymopoeitin, thymosin, thymostimulin, thymic factors, bombesin, neurostensin, lysozyme, protein synthesis stimulating peptides, vasoactive intestinal polypeptide, growth hormone releasing factor, and somatocrinin.

Certain fish and other marine animals contain oil rich in long chain polyunsaturated fatty acids (LC-PUFAs), such as eicosapentaenoic acid (EPA) and docosahexaenoic acids (DHA). Since these fatty acids have a double bond between the third and fourth carbon from the terminal methyl group of the fatty acid, they are referred to as omega-3 fatty acids. The positive health effects of consuming fish oil containing omega-3 fatty acids have been widely reported in recent years (Harris et al., 2003, Circulation 107:1834-1836; Kyle, 2002, Essential Fatty Acids as Food Additives, in Food Additives, 2nd ed., Branen et al., Eds., Marcel Dekker Inc., New York, pp. 277-310; Kyle, 2002, The Role of DHA in the Evolution and Function of the Human Brain, in Brain Lipids and Disorders in Biological Psychiatry, Skinner, Ed., Elsevier Press, Amsterdam, pp. 1-22; Kyle, 2001, The Large Scale Production and Use of Single-Cell Oil Highly Enriched in Docosahexaenoic Acid, in Omega-3 Fatty Acids; Chemistry Nutrition and Health Effects, Shahidi et al., Eds., Oxford Press UK, p. 354; Conner, 2000, Am. J. Clin. Nutr. 71:171S-175S). These positive health benefits have been seen in humans and in animals.

Dietary sources of omega-3 LC-PUFAs can be found mainly in foods prepared from marine sources such as algae and fish. In most populations, however, the nutritional benefits of PUFA compounds cannot be realized due to the low consumption of fish and edible algae. With the U.S. Food and Drug Administration's current allowance for health claims relating to intake of omega-3 fatty acids for protection from heart disease, there is an increased interest in fortifying food products with these components. One main problem that hinders the incorporation of omega-3 PUFA oils into processed foods is the high degree of unsaturation, resulting in its susceptibility to oxidation and the subsequent deteriorative effects on flavor and aroma profiles of the oil. Gelatin capsules containing fish oil with omega-3 fatty acids have been available to consumers for some time (Jizomoto et al., 1993, Pharm. Res. 10(8):1115-1122). In recent times, efforts to incorporate fish oils containing omega-3 fatty acids into general food products have occurred (Yep et al., 2002, Asia Pac. J. Clin. Nutr. 11(4):285-291; Wallace et al., 2000, Ann. Nutr. Metab. 44(4):157-162). The food products consist of beverages, salad dressings, mayonnaise, yogurt, ice cream, cookies, cakes, and processed meats. The particles described herein are suitable for delivering such oils, lipids, and fatty acids to animals.

Dissoluble Lipid

Certain particles described herein (e.g., microparticles) include a lipid that is dissoluble in at least one compartment of the animal. The identity of the lipid is not critical, in that it need only function as a barrier to inhibit water on the outside of the particle from contacting the bioactive agent in the interior of the particle under normal storage conditions. The dissoluble lipid should not (or should to a much lesser degree) inhibit such contact when the particle is in the desired compartment of the animal. By way of example, the dissoluble lipid can be one that is solid at a temperature lower than the temperature of the compartment, one that is more soluble at the pH of the compartment than at another pH, or both.

Examples of lipids that are solid at low temperatures but liquid at higher temperatures include both natural and synthetic oils. Examples include animal fats, such as lard, butter, and alcohol esters of polyunsaturated fatty acids or fractions thereof, vegetable fats such as cocoa butter, cocoa butter equivalents, olive oil, palm oil, palm kernel oil, or fractions thereof, hydrogenated oils, microbial oils, algal oils, yeast oils, fungal, alcohol esters of polyunsaturated fatty acids, natural waxes, alcohol esters, cholesterol esters, phytosterol esters and solid mineral oils such as paraffin and mixtures or fractions of these. The advantage of using solid fats or polyunsaturated fatty acid waxes for this purpose is that their physical properties can be tailored to the properties of the active agent and the desired use. This can be done by manipulation of the fatty acid composition, and is understood in the art. The carbon chain length of the fatty acid affects the melting point of the ester (i.e., melting points increase with increasing molecular weight and degree of unsaturation of the fatty acid).

Examples of suitable animal oils and fats include: beef tallow (which has a melting point of about 35-38 degrees Celsius), mutton tallow (which has a melting point of about 40-45 degrees Celsius), wool fat and grease, butter, cholesterol esters, stearine (which has a melting point of about 49-55 degrees Celsius) and stearic acid (which has a melting point of about 71 degrees Celsius). Solid vegetable oils include: hydrogenated oil, coconut oil, coconut butter, olive oil, palm oil, palm kernel oil, castor oil, linseed oil, soybean oil, cocoa butter, cocoa butter equivalents, and phytosterol esters. Solid fish oils include: cod oil, herring oil, salmon oil, sardine oil, jap fish oil, menhaden oil, whale oil, sperm oil. Natural waxes include: carnauba wax (which has a melting point of about 78-81 degrees Celsius), candelilla wax (which has a melting point of about 68 degrees Celsius), beeswax (which has a melting point of about 60-63 degrees Celsius), permaceti-sperm oil (which has a melting point of about 42-49 degrees Celsius), Japan wax, jojoba oil and hardened jojoba oil and wool fat and grease (which has a melting point of about 30-40 degrees Celsius). Hydrocarbons (unsaponifiable) include: paraffin wax (which has a melting point of about 35-36 degrees Celsius), montan wax (which has a melting point of about 76-84 degrees Celsius), ceresine wax (which has a melting point of about 60-85 degrees Celsius). The final melting point of lipids can be manipulated through mixing two or more lipids of different melting points. Liquid oils can be converted into solid fats in about room temperature through hydrogenation. Natural waxes, such as bees wax, carnauba wax, candelilla wax, spermaceti wax, Japan wax, jojoba oil, and hardened jojoba oil, can be used either alone or in a mixture with other liquid or solid lipids provided that the final melting point of the solid lipid is retained at above the temperature that the particles or a product containing the particles is maintained.

Emulsifier

Emulsifiers are known in the art, and substantially any emulsifier can be used in a particle described herein. Examples of suitable emulsifiers include monoglycerides, sorbitan esters, propylene glycol esters, phospholipids, lecithins, polysorbates, sucrose esters of medium chain saturated fatty acids (e.g., having an acyl group containing more than about 10 carbon atoms), sucrose esters of long chain saturated fatty acids, (e.g., saturated fatty acids which contain from about 12 to about 18 carbons), sucrose esters of unsaturated fatty acids (e.g., unsaturated fatty acids which contain from about 12 to about 22 carbons, such as oleic, linoleic, EPA, ARA and DHA).

Dissoluble Coatings

The particles described herein can be wholly or partially contained within a coating. In order to deliver the bioactive composition of the particle to a compartment of an animal, the coating should be dissoluble in at least that compartment. Coatings that are dissoluble in one compartment of an animal preferably over another compartment (or which are dissoluble in one compartment but substantially indissoluble in another) are known in the art and are also suitable for coating the particles described herein.

Examples of dissoluble coatings are coatings made of materials such as amylopectin, waxy maize starch, soluble starch, gluten, casein, albumin, fishmeal, fishmeal hydrolysate, krill meal, shrimp meal, soy meal, wheat meal, cotton seed meal, and pea meal. Many other coatings are known in the art. Coatings that can be digested by the animal to which the particle is administered can be used, but it is not necessary that the coating be digestible or that it have any nutritive value to the animal.

Other Ingredients

The particles described herein (or portions of the particles, such as a coating) can include one or more additional ingredients intended to enhance the flavor, appearance, or other characteristics of the particles, even if the additional ingredient is biologically inert (i.e., it is not a “bioactive agent”). Examples of such ingredients can include pigments, foaming agents, viscosity regulators, flavorants, flavor-stabilizing agents, preservatives, fillers, bulking agents, and the like.

The particles may have a bioadhesive agent associated with them (e.g., beneath an outer coating). The advantage of using a bioadhesive suitable for adhesion of the particles to a mucosal surface, such as that of the intestinal tract is that such bioadhesive particles will persist longer in the system, especially if the microparticles are degrade slowly. Bioadhesive particles may be particularly suitable for the oral administration of bioactive agents to relatively poorly-developed digestive systems, such as those of young animals and fish. Examples of suitable bioadhesives are hydrocolloids such as chitosan, cyclodextrins, phenylalanine enamine of ethylacetoacetate, diethyleneoxymethylene malonate.

The particles described herein can also include a permeability modulating agent, such as one that increases or decreases the permeability of a membrane lining a compartment of the animal to or through which the particles are delivered. Permeability modulating agents are known in the art. Examples of suitable agents include water-soluble phospholipids, lysophosphatidylcholines such as those produced from egg or soy lecithin, acyl glycerols, fatty acids and salts, acyl carnitines (e.g., palmitoyl-DL carnitine-chloride) and biological detergents (such as bile salts and analogues). Other biological agents and surfactants that modify the intestinal mucosal membrane fluidity and permeability can also be used.

The substantially indigestible polymer matrix of the particles described herein can include a digestible or dissoluble component that increases the porosity or permeability of the matrix or that lessens the cohesion or strength of the matrix when the particle is in a compartment of the animal, such as the compartment to which the bioactive agent is to be delivered. Such components can enhance the rate or degree of release of the bioactive agent from the matrix. Many such components are known in the art, and a skilled artisan is able to select an appropriate component based on the nature of the matrix and the compartment of the animal and the desired effect on the rate or degree of release.

Uses

The particles described herein can be used to deliver substantially any composition of matter that can be accommodated by the particle to a compartment of an animal. In an important embodiment the particles are intended for oral administration, in order to deliver a bioactive agent to a compartment (e.g., the stomach, small intestine, large intestine, or some combination of these) of the gastrointestinal system an animal. However, it is recognized that the utility of the particles described herein is not limited to oral or gastrointestinal applications. The particles can be delivered to one or more other compartments of an animal (e.g., the interior of the lungs, the pleural sac, the peritoneum, the space within the ear canal, or the periocular space) in order to deliver a bioactive agent to those compartments.

The particles described herein can be administered alone, or as a component of another composition. In the context of orally-administered compositions, the particles can be administered as a dry powder, a wet powder or paste, a suspension of particles in a liquid, a tablet, a capsule, or an ingredient in a food product, for example. The particles can be incorporated into foods intended for special purposes, such as performance foods, mood foods, medical foods, nutritional snacks or supplements, sport foods (e.g., power bars), baby foods, toddler foods, infant foods, or foods intended for pharmaceutical or dietetic purposes. The microparticles of the present invention may be used as or incorporated into a topping for breakfast cereals, snacks, soups, salad, cakes, cookies, crackers, puddings, desserts, or ice cream. They may also be used as an ingredient for yogurts, desserts, puddings, custards, ice cream or other pasty or creamy foods. Regularly sized pieces may be individually packaged or used as nutritional snacks or, for example, added to or formed into nutritional food in bars.

The particles described herein as microbeads are suitable for incorporation into foods products that are cooked after incorporation of the microbeads. By way of example, microbeads can be incorporated into foods intended for human or animal consumption, such as baked goods (e.g., bread, wafers, cookies, crackers, pretzels, pizza, and rolls), ready-to-eat breakfast cereals, hot cereals, pasta products, snacks (e.g., fruit snacks, salty snacks, grain-based snacks, and microwave popcorn), dairy products (e.g., yogurt, cheese, and ice cream), sweet goods (e.g., hard candy, soft candy, and chocolate), beverages, animal feed, pet foods (e.g., dog food and cat food), aquaculture foods (e.g., larval diets, enrichment diets, fish food and shrimp feed), and special purpose foods (e.g., baby food, infant formulas, hospital food, medical food, sports food, performance food or nutritional bars), or fortified foods, food pre-blends or mixes for home or food service use (e.g., pre-blends for soups or gravy, dessert mixes, dinner mixes, baking mixes, bread mixes, cake mixes, and baking flour).

The particles described herein can be used to deliver oils, bioactive agents, or other compositions of matter to substantially any animal able to ingest the particles, including both aquatic animals and terrestrial animals. Aquatic animals include, but are not limited to, crustaceans, rotifers, mollusks, elasmobranchs, teliosts, and aquatic mammals. Terrestrial animals include, but not limited to, sheep, goats, cattle, horses, pigs, mice, rats, guinea pigs, dogs, cats, birds, and reptiles, and humans.

Manufacture

The particles described herein can be made in a variety of ways that will be apparent to skilled artisans in this field. Methods described in this section for making such particles are examples only. The method used to make the particles described herein is not critical.

Preparation of Microparticles

The microparticles disclosed herein can be produced by blending a bioactive agent and a molten dissoluble lipid. The mixture is solidified by reducing the temperature of the mixture to below the melting point of the lipid, preferably while spraying rapidly stirring, or emulsifying the mixture to form cooled particles in which at least the lipid is solid. Alternatively, a solid mass of the lipid and agent can be formed and milled, cut, or otherwise divided to form particles therefrom.

Solid lipid/agent particles are thereafter incorporated into a substantially indigestible polymer matrix, together with any other desired ingredients. In one method, a precursor of the polymer matrix is suspended or dissolved in a liquid to which the solid lipid/agent particles are added, while the liquid is maintained at a temperature lower than the melting point of the lipid. The precursor is polymerized or cross-linked to form the matrix, within which the lipid/agent particles are entrapped.

In another embodiment, the bioactive agent and molten solid lipid blend are emulsified with water (in a ratio of about 2 parts water to about 1 part of solid lipid) containing 0.1-10% emulsifier while maintaining the temperature of the emulsion at or above the melting point of the solid lipid. The emulsion is cooled to below the melting point of the solid fat and then admixed with a precipitatable and/or insoluble hydrocolloid to form a substantially indigestible matrix around or including portions of the emulsion.

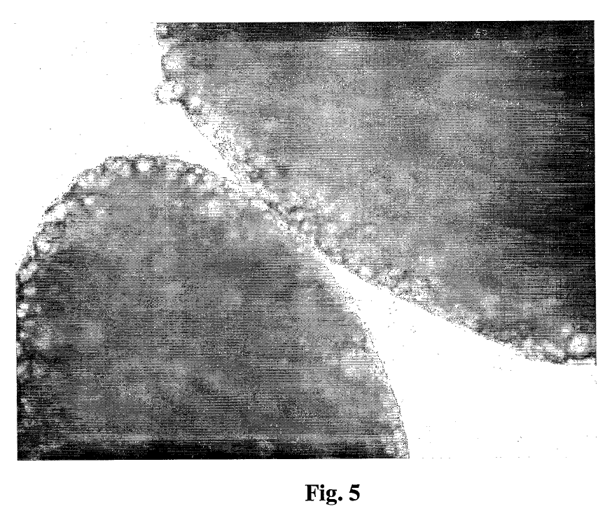

In yet another embodiment, the bioactive agent and molten solid lipid are blended and emulsified together with a precipitatable and/or insoluble hydrocolloid (in a ratio of about 2 parts of precipitatable and insoluble hydrocolloid gel to 1 part of solid lipid) containing around 0.1-10% emulsifier while maintaining the temperature of the slurry at or above the melting point of the solid lipid. The slurry is then sprayed or dropped into a chilled and/or low pH bath or a solution containing about 0.1-20% divalent cation or metal. As illustrated in FIG. 5, alginate matrix microparticles loaded with solid lipid droplets containing the bioactive agent can be made in this way.

As another example, a precipitatable hydrocolloid such as an alginate can be dispersed in a water solution in an amount of about 1 to 25% w/w at a temperature range of about 20 to 90 degrees Celsius until a uniform and viscous gel is obtained. Any desired additional ingredients, such as preservatives, digestible materials, permeability releasing agents, pigments, flavorants, or the like can be added to the gel mixture. Lipid/agent or an emulsion of lipid and agent in water, for example, are mixed with the hydrocolloid at a ratio of about 0.1%-25% of the bioactive agent. The slurry is then internally cross-linked by the addition of calcium ion (e.g., calcium chloride). The slurry can instead be dripped, injected, or atomized through a nozzle into a chilled 0.1% to 20% solution of calcium-chloride (preferably at a pH of 2-5) in water. The cross-linking can be permitted to continue about 5 to 60 minutes. In a method such as this, the preferred solvents for the solution of multivalent cations are water and/or a low molecular weight alcohol, such as methanol, ethanol, or isopropyl alcohol. Higher molecular weight alcohols may also be used, but the low molecular weight alcohols are preferred because they can be removed more easily from the microparticles by volatilization. In general, water is the preferred solvent. However, if the bioactivity of the microparticle is not damaged by the use of an organic solvent, alcohol is then the preferred solvent because it precipitates the gel matrix and is also easily volatilized.

A bulk insoluble gel can be chopped, ground, or milled, while still wet to form small beads or particles. Particles formed by spraying or dripping into a cross-linking bath can be readily harvested and sorted into various sizes. If desired, the particles can be refrigerated (e.g., at 4 degrees Celsius) until use. Optionally, the particles can be dried to produce a powder by a number of methods recognized in the art, including low temperature spray drying, belt drying, freeze drying, vacuum drying, drum drying, or flash drying. The dried particles can be stored at cold or at elevated temperatures. Dried microparticles can be rehydrated with water or another aqueous medium prior to use or allowed to rehydrate on delivery. Dried materials can also be further milled and sieved to produce smaller particle sizes.

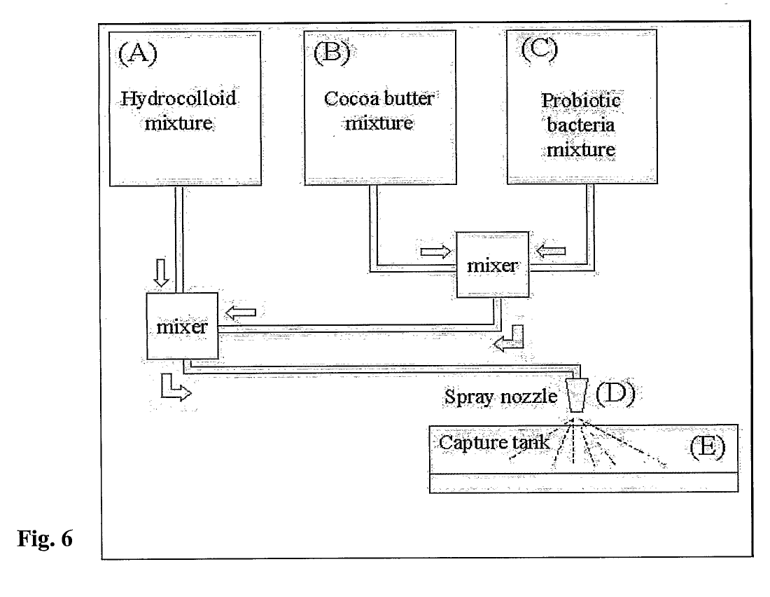

In an embodiment illustrated in FIG. 6, a hydrocolloid matrix material is prepared in advance as a composition of gelatinized high amylose starch, lecithin, and alginate as described by Hard (International Patent Application publication no. WO 2004/043140) and is maintained in a vessel (A) at a temperature of around 40 degrees Celsius. In a second vessel (B) a stock of cocoa butter is maintained in the liquid state at around 40 degrees Celsius. In a third vessel (C) is a powdered and preserved form of Lactobacillus sp., that is maintained at less than 20 degrees Celsius. The dry material from (C) is metered into the stream of molten cocoa butter from (B) and mixed in an in-line mixer. This stream is then mixed with the output from (A) also in an in-line mixer. The resulting emulsion is maintained at (C) but immediately passed through an atomizing nozzle (D) forming particles of about 50-250 micrometers in diameter. The particles are then captured in a tank (E) containing a 1% calcium chloride solution maintained at less than around 20 degrees Celsius. Particles are continuously harvested from this tank, rinsed with fresh water, and flash frozen prior to drying so that the overall exposure time of the particles to the calcium chloride is less than about 15 minutes. This results in the simultaneous cross-linking of the alginate and solidification of the cocoa butter. The overall process can limit the time of exposure to and elevated temperature to less than around 1 minute and the exposure time to water to about 15 minutes.

Preparation of Microbeads

Microbeads such as those disclosed herein can be produced by gelatinizing high amylose starch containing at least 50% amylose. Numerous methods of gelatinization of starch are well known in the art, including direct or indirect heating of an aqueous dispersion of starch, by chemical treatment of such dispersion using strong alkali, or a combination of mechanical, chemical and heat treatment. Normally, a skilled artisan would expect that the gelatinization of starch is undesirable to obtain a formulation suitable for gastric protection. However, in accordance with the instant invention, it has been unexpectedly found that the addition of an emulsifier in a ratio of from 1 part emulsifier to from 0.5 to 10 parts starch, and preferably from 1 part emulsifier to from 3 to 5 parts starch, causes the swollen starch granules to dissociate and the free amylose polymers are believed to form a complex with the emulsifier. This complex of high amylose polymers and emulsifier, which is soluble in a slightly alkali solution, permits the admixing of large quantity of oil-soluble materials and enhances the gastric-resistance properties of the composition.

In a preferred embodiment of this invention, from 1% to 25% w/w of high amylose starch (e.g., at least 50% amylose) is dispersed in a basic solution (e.g., 0.2-5 normal sodium hydroxide solution) at a temperature of from 20 to 65 degrees Celsius until starch granules are fully expanded. Alternatively, a modified high amylose starch can be used without the need for the basic solution.

Other matrix components, such as soluble starch, proteins, and polypeptides can be added to increase the rate of release of the bioactive agents from the finished microbeads. Examples of possible materials that could be used for modulating the rate of release include fructose, sucrose, pectin, whey proteins, casein, albumen, soy proteins, fishmeal, and krill meal. These rate-increasing components can dissolve more readily in water and gastric juices than amylose based matrix material. Upon dissolution, permeability of the microbeads is increased, thereby increasing access to the bioactive agent(s) in the microbead.

Once the high amylose starch is gelatinized an emulsifier can be added in a ratio of 0.1 to 2 portions emulsifier per portion of the starch, and more preferably in a ratio of 0.1 to 1 portions emulsifier per portion of starch. The temperature is maintained in the range of 20 to 65 degrees Celsius until the starch granules are completely dissolved and a slurry complex is completely soluble and stabilized by the interaction between the amylose polymers and emulsifier.

The alkalinity of the product is slowly adjusted to pH 7.5-8 by addition of acid. The starch and emulsifier complex can also be co-processed with other hydrocolloids, gums, polymers, modified starches, and combinations of these to change the water binding capacity of the starch-emulsifier compositions. For example, xanthan gum, alginate, carrageen, carboxymethyl cellulose, methyl cellulose, guar gum, gum Arabic, locust bean gum and combinations thereof can be added to the starch-emulsifier compositions at any time after the pH neutralization, as long as the additional ingredient(s) do not disrupt the formation of the amylose-emulsifier complex. In the case of some hydrocolloids and starches, it may be possible to eliminate the emulsifier completely. The slurry composition is allowed to cool down to room temperature.

Oil-associated bioactive compounds are mixed into the slurry either alone or in a mixture of other bioactive agents in an amount of from 0.1% to 60% of slurry. Any type of preservative such as, but not limited to, propylene glycol, glycerol, or BHT, can be added if desired. The slurry is then cross-linked by the addition of a solution of calcium salts (e.g., Calcium chloride, Calcium sulfate, Calcium acetate) to the slurry or by dripping, injecting, or atomizing the slurry through a nozzle into an solution containing 10 millimolar to 1,000 millimolar calcium ions (e.g., 0.1% to 10% of CaCl₂) and allowing the particles to cross-linked for about 5 to 60 minutes.

Excess calcium chloride can be removed by a washing procedure, which may include several washing steps. The first washing solution may contain surfactants and/or soluble polymers that are cross linked in acid conditions such as polysaccharides or gums, followed by an acid wash and by rinsing with tap water.

The wet solid gel can then chopped into small beads or the atomized or dripped microbeads are harvested from the cross-linking medium. The resulting material can be sorted into various sizes and stored until use. The microbeads can optionally be dried to produce a powder by a number of methods recognized in the art, including low temperature spray drying, belt drying, freeze drying, vacuum drying, drum drying, or flash drying. Dried microparticles can be rehydrated with water or another aqueous medium prior to use or allowed to rehydrate on delivery.

### EXAMPLES

The invention is now described with reference to the following Examples. These Examples are provided for the purpose of illustration only, and the invention is not limited to these Examples, but rather encompasses all variations which are evident as a result of the teaching provided herein.

### Example 1

Preparation of High Amylose Starch Phospholipid and Alginate Complex Slurry

Two grams of high amylose starch (HYLON™ VII, National Starch and Chemical, Bridgewater, N.J.) is dissolved in 96 milliliters of 1% sodium hydroxide at 50 degrees Celsius. One gram of powdered egg lecithin (Archer-Daniels-Midland Co., Decatur, Ill.) or liquid soy lecithin is added to the alkali slurry and allowed to dissolve the hydrated starch granules and to complex with the amylose polymers for 30 minutes. The alkali complex slurry is then neutralized to pH 7.5 with hydrochloric or acetic acid, 1 gram alginate (PRIME ALGIN™ T-500, Multi-Kern Corp., Raidefield N.J.) dissolved into the slurry and cooled to room temperature. The slurry is now ready for the addition of oil or oil associated bioactive agents and to be cross-linked to calcium ions. The composition of the complex slurry is provided in Table 1.

TABLE 1
Slurry composition (grams dry weight per 100 grams)

| | |
|---|---|
| High amylose (70% amylose) | 2 |
| Egg/soy lecithin | 1 |
| Alginic acid | 1 |
| Water | 96 |

### Example 2

Fish Oil-Containing Microbeads

1000 milliliters of complex slurry is prepared according to Example 1 and 200 grams of fructose (the Estee Company garden city, N.Y.) and (400 grams) of fish oil was mixed into the solution. The fish oil contained 200 parts per million of tertiary butylhydroquinone (TBHQ) and 1,000 parts per million of tocopherols and/or 0.5% rosemary oil. The preferred fish oil is refined and deodorized and contains a high quantity of omega-3 fatty acids. The fish oil of the present invention may be produced from any suitable source, including sardines, herring, capelin, anchovy, cod liver, salmon, tuna, and mixtures thereof. Acceptable particles have also been prepared in the absence of fructose.

To mask any fishy flavor and smell, sensory masking agents such as vanillin or natural and artificial fruit or mint flavors such as lime, lemon, orange, pineapple, grapefruit, spearmint, peppermint, benzaldehyde, and cherry, may be included at this stage. The slurry is then atomized through a nozzle into a water bath containing 2% calcium acetate and 2% pre-dissolved gelatin. The microparticles range from 10 micrometers to 600 micrometers in diameter, and are harvested using a fine mesh screen (68 micrometers) and gently rinsed with citric acid containing water (pH 4.5). The wet microparticles will contain about 40% by wet weight of fish oil. Conventional drying of these particles lead to a total lipid content of 97% lipid.

Microbeads loaded with fish oil can also be made in a dry form using an alternative approach. In this case the initial slurry composition consists of 2 grams of high amylose starch, 1 gram of lecithin, 1 gram of alginate, and from 2 grams PUFA oil (fish oils, microbial oils, vegetable oils or any combination thereof) and 94 grams water. This slurry was atomized into a calcium chloride bath as described above, but it can also be internally set by the rapid mixing with dilute CaCl₂ or slow release calcium ion and pouring the mixture into a setting mold. The atomized particles (or chopped and diced internally set material) were then vacuum-dried at room temperature. The resulting particles can be used “as-is” or milled and sifted to a specific size range between 5 microns and 5,000 microns. The oil content of the resulting dried material from atomized particles was found to be 50% by dry weight following extraction of the dry material with hexane and weighing the hexane extract. The high lecithin to oil ratio (1:2) also provides an unexpectedly high degree of oxidative stability to the powder. Monosaccharide such as, but not limited to fructose, can also be added to the mixture at from 1 to 40 grams per 100 grams mixed slurry to provide additional structural stability to the dried particles. Oil loads from 10% to 70% can be routinely obtained by adjusting the amount of starting oil.

Sonication Test

To test for relative strength, the microbeads are sonicated. Ten milliliters of water and 0.1 gram of microbeads are blotted using a paper towel. After standing for 5 minutes, microbeads are sonicated using a BRANSONIC™ cell disruptor model 185 (Danbury, Conn.) for 2 minutes. Ultrasonic treatment breaks the microbeads, releasing the oil. The oil release was indicated by increased turbidity in the aqueous phase. The turbidity of the aqueous phase was measured by a spectrophotometer at 595 nanometers. Higher turbidity indicated more broken capsules, therefore, more fragile and unstable microbeads.

Heating Test

This test provides relative values on the thermal stability and mechanical strength of the microbeads. A small amount of microbeads was spread on a glass microscope slide and dried at 50 degrees Celsius overnight and weighed. Then the sample was then heated at 265 degrees Celsius for 20 minutes and weighed again. The amount of oil loss is recorded by calculating the weight difference between the two measurements.

The microbead product will be stable for at least 2 months with only minor evidence of fishy odor.

### Example 3

A Yogurt Food Product Containing Microbeads

Microbeads containing algal oil were prepared according to Examples 1 and 2 except that the fish oil was replaced with 400 grams of algal source DHA oil (DHASCO™, Martek, Columbia Md.). The resulting wet beads will be 40% by weight oil and about 20% by weight DHA. A yogurt composition is prepared by admixing 100 grams of DANNON™ brand plain, low fat yogurt with 2.5 grams of the above microbeads. The final yogurt product contains 400 milligrams of DHA per 100 grams yogurt and has no evidence of fishy odor or flavor.

### Example 4

A Mayonnaise Containing Microbeads

Fish oil containing microbeads were prepared according to Example 2 followed by a sieving into 2 size groups of microbeads (above 150 micrometers and below 150 micrometers). A mayonnaise composition is prepared by admixing 90 grams of Hellmann's brand real mayonnaise with 10 g of the small size microbeads (below 150 micrometers). The final mayonnaise product contains 2000 milligrams of DHA per 100 grams mayonnaise and has no evidence of fishy odor or flavor.

### Example 5

An Infant Formula Containing DHA and ARA Oils Microbeads

Microbeads are prepared in a dry format according to Examples 1 and 2 using DHA and ARA oils (DHASCO™ and ARASCO™, Martek, Columbia Md.). The wet microbeads are first sieved into 2 size groups of microbeads (above 50 micrometers and below 50 micrometers) using a vibrating screen device, and then vacuum dried. An infant formula is prepared by admixing 99 grams of Enfamil (Mead Johnson) with 1 gram of the small size, dried microbeads (below 50 micrometers). The final product contains 200 milligrams of DHA per 100 grams infant formula.

### Example 6

Probucol and S-312-d, a Calcium-Channel Blocker, are Employed as Model Lipophilic Drugs

Glyceryl tricaprylate and tricaprate mixture solutions containing these drugs are admixed in complex slurry according to Example 1 and recovered as free-flowing powders as in Example 2. Microbeads are stored as a powder at room temperature in a closed bottle, with no significant change in appearance or disintegration time upon rehydration observed even after 1 year. Oral bioavailability is tested in rats and compared with those from other conventional formulations. Gastrointestinal absorption of both Probucol and S-312-d from the microcapsules will be more efficient than that from other formulations such as powders, granules, or oil solution.

### Example 7

The Bactericides Triclosan and Chlorhexidine are Employed as Model Lipophilic/Hydrophilic Antibiotic System

A soy oil solution containing 100 parts per million of triclosan is admixed in a complex slurry containing 100 parts per million of chlorhexidine according to Example 1. Microbeads are then produced and recovered as free-flowing powders as in Example 2. Microbeads are stored as a powder at room temperature in a closed bottle with no significant change in appearance or disintegration time upon rehydration even after 1 year. Oral bioavailability is tested in rats and compared with those from other conventional formulations. Gastrointestinal absorption of both triclosan and chlorhexidine from the microbeads will be more efficient than that from other formulations such as powders, granules, or oil solution.

### Example 8

Liposomes of Dipalmitoylphosphatidylcholine (DPPC) Containing Acetylsalicylic Acid (ASA)

Liposomes of dipalmitoylphosphatidylcholine (DPPC) containing acetylsalicylic acid (ASA) are added at a level of 40% to the complex described in Example 1. Microbeads are then produced as in Example 2. If dry particles are preferred, the DPPC component is only added to 5% of the complex described in Example 1, the microparticles are dried, and recovered as free-flowing powders for a potential oral drug delivery system. The stability of the microbeads containing liposomes in sodium cholate solutions at pH 5.6 will be much greater than the corresponding liposomes.

### Example 9

Microbeads for Fish and Crustacean Larvae

A microbead slurry containing 20% fish oil and 20% Chlorella sp., Nanochloropsis sp. or Tetraselmis sp. algal biomass and/or 10% fish meal (on a dry weight basis) is prepared according to Example 1. The slurry then atomized through a nozzle into a water bath containing 2% calcium acetate as in Example 2. The microbeads with a size distribution between 50-200 micrometers are harvested using a vibrating sieve and gently rinsed with fresh water. The wet microparticles are then vacuum dried or stored wet in an air-tight container at 4 degrees Celsius for delivery to fish or shrimp larvae. Feed grade preservatives can then be added to the wet beads prior to packaging for prolonged shelf life.

### Example 10

Feeding of Shrimp (Penaeus vannamei) with a Fish Oil and Probiotic Mixture

A microbead slurry containing 0.2% L. rhamnosus bacteria and 40% fish oil is prepared as described in Example 1 and wet microbeads are prepared as in Example 2. Shrimp fry at about 1.0 gram size are stocked at 10 kilograms per cubic meter of seawater at 28 degrees Celsius. Water quality is maintained by rapidly exchanging the tank water through mechanical and biofiltration systems. Shrimp are fed a standard pelleted feed 4 times daily a total ration of 2% body weight with pellet size adjusted to fit the mouth opening of the growing shrimp. In addition to the standard feed, shrimp are fed with 0.2% body weight of wet microbeads (2000 micrometers in diameter) described above. Shrimp grown under such conditions exhibit an increased growth rate (final weight minus initial weight divided by the duration of the experiment), increased food conversion ratio (total food provided divided by the total final biomass minus total initial biomass), and/or increased resistance to viruses such as White Spot Syndrome Virus (WSSV).

### Example 11

Microbeads of the Instant Invention Containing Carotenoids for Coloring Salmon and Trout Flesh

A microbead slurry is prepared according to Example 1 with the addition of (40 grams per 100 grams slurry) of Haematococcus algae containing natural astaxanthin (NATUROSE™, Cyanotech Corporation, Kailua-Kona, Hi.). After thorough mixing for about 1 hour, the mixture is atomized as described in Example 2 and the wet microbeads are harvested. About 100 grams of wet beads are dissolved in 1000 milliliters of Menhaden oil (Omega Protein, Houston, Tex.) and used to top-coat 1 kilogram of feed pellets. The resulted feed contains 40 milligrams astaxanthin per kilogram feed and can be fed to salmonid fish for coloring of the flesh. Alternatively, the wet microbeads, or a dried form thereof can be incorporated directly into the feed mixture prior to extrusion and/or pelleting.

### Example 12

Feeding Oysters with Microbeads Containing a Mixture of Fish Oil and Probiotic Bacteria

Microbeads are prepared as described in Example 1, 2 and 10, and air-dried. Oysters spat (Crassostrea gigas) are stocked in larval rearing system at a density of 100 per liter in full seawater (32-40 parts per thousand) at 25-29 degrees Celsius. Oysters are given a daily mixture of the live algae Tetraselmis sp. and Chaetoceros sp., at concentrations of 10,000 and 5,000 cells per milliliters, respectively and with 5 milligrams per liter of air dried microbeads until 40 days post-hatch. Tanks are then harvested and counted individually for survivorship and sampled for average weight.

### Example 13

Feeding Cats with Extruded Feeds Containing Fish Oil Microbeads

A dry microbead preparation containing 50% by weight fish oil and 10% by weight ARA-oil (Martek Biosciences Corp) is prepared according to Examples 1 and 2 and added to a standard commercial cat feed mixture at a level of 2% (w/w). The mixture is then extruded and the resulting pellets will contain about 0.2% EPA+DHA and 0.1% ARA.

### Example 14

Feeding Laying Hens with Beadlets Containing Fish Oil

The slurry according to Example 2 and dry powdered Lactobacillus acidophilus are blended to obtain a substantially homogeneous dry blend. The dry blend and water are separately fed into a feed port of a Werner & Pfleiderer twin screw extruder at a total rate of about 2.5 kilograms per hour. The pressure at the extruder inlet is atmospheric. All barrels of the extruder are kept at a barrel temperature of about 21 degrees Celsius. The extruder die consists of 40 circular openings, each 0.5 millimeter in diameter. Upon exiting the die, the exiting ropes are cut with rotating knives into discrete particles of 0.5-1.5 millimeter length and allowed to cross-link in a water bath containing 3% CaCl₂. The beadlets are harvested and dried for about 30 minutes either in a vacuum drier or under CO₂ or another inert gas to prevent oxidation in order to produce shelf-stable pellets which contain encapsulated fish oil and protected, active, live microorganisms.

Sixteen laying hens at size of about 500 grams are housed in windowless sheds at a stocking density of 20 kilograms of bird weight per square meter. Temperature and ventilation are automatically controlled. Hens are fed a standard commercial diet 4 times daily at a total ration of 4% body weight. Hens are also fed with 1% of daily ration with the above microbeads. Eggs are collected for a period of 4 weeks following the probiotic and fish oil feeding treatment and are analyzed for Salmonella contamination and DHA and EPA content.

### Example 15

Feeding Swine with Feed Containing Microbead with Fish Oil

A standard commercial swine feed is amended with 1% of microbeads (dry weight) containing 50% fish oil loaded dry microbeads from Examples 1 and 2. The mixture is then pelleted with an extruder and fed to 50 pigs at age 3-5 weeks. Survival and growth rates were monitored.

### Example 16

Preparation of Microbeads Containing DHA Algae

Microbeads are prepared as in Examples 1 and 2, except the slurry composition consists of 20 grams of fish oil and 20 grams (dry weight) of Schizochytrium biomass (Advanced BioNutrition Corp). Wet microbeads produced by this process provide excellent supplemental feeds for larval aquatic animals (fish and crustaceans) when provided directly or in combination with rotifers and/or artemia. Microbeads can also be provided in a dry form by vacuum drying the wet beads. In the dry form the beads comprise about 40%-45% oil and 40%-45% algal biomass.

### Example 17

Preparation of High Amylose Starch and Alginate Complex Slurry

Two grams of high amylose starch (HYLON™ VII, National Starch and Chemical, Bridgewater, N.J.) is dissolved in 96 milliliters of 1% sodium hydroxide at 50 degrees Celsius. The alkali slurry is then neutralized to pH 7.5 with hydrochloric or acetic acid, 1 gram alginate (PRIME ALGIN™ T-500, Multi-Kern Corp., Raidefield N.J.) dissolved into the slurry and cooled to room temperature. The slurry is now ready for the addition of oil or oil associated bioactive agents and to be cross-linked to calcium ions. The composition of the complex slurry is provided in Table 2.

TABLE 2
Slurry composition (grams dry weight per 100 grams)

| | |
|---|---|
| High amylose (70% amylose) | 2 |
| Alginic acid | 1 |
| Water | 96 |

### Example 18

Probiotic-Containing Microparticles

Preparation of Cocoa Butter—Probiotic Emulsion

Pure cocoa butter (100 grams) was melted in a microwave then maintained at 36 degrees Celsius. An equal amount of a dry powder of Lactobacillus acidophilus GG (100 grams Valio, Finland) was blended with the molten cocoa butter, using a kitchen blender, while maintaining the temperature at 36 degrees Celsius. Warm distilled water with 0.1% TWEEN® 80 (200 milliliters at 36 degrees Celsius) was immediately added to the blender and the mixture emulsified for 1 minute. Crushed ice was then added, while continuing to blend, to reduce the emulsion temperature to 20-25 degrees Celsius and to solidify the cocoa butter microdroplets containing the probiotic bacteria.

Preparation of Hydrocolloid Solution

Alginate (1% Prime Algin T-500, Multi-Kem Corp., Raidefield N.J.) was dissolved in 700 milliliters of distilled water at room temperature, using a kitchen blender and maintained at room temperature.

Preparation of Probiotic Microparticles of the Present Invention

The solidified cocoa butter probiotic emulsion was blended into the alginate solution using a kitchen blender while maintaining the temperature below the melting point of the cocoa butter. The slurry was then atomized into a 1-2% w/w calcium chloride bath using a commercially available paint sprayer to form microparticles in a size range between 10 micrometers and 600 micrometers. The microparticles were harvested from the calcium chloride bath by filtration, rinsed with fresh water then freeze-dried. The dry powdered microparticles were packed under nitrogen in humidity-resistant foil bags.

The composition of the microparticle slurry is provided in Table 3.

TABLE 3
Slurry composition (grams per 100 grams)

| | |
|---|---|
| LGG dry powder | 10 |
| Cocoa butter | 10 |
| Alginate | 1 |
| Water | 79 |

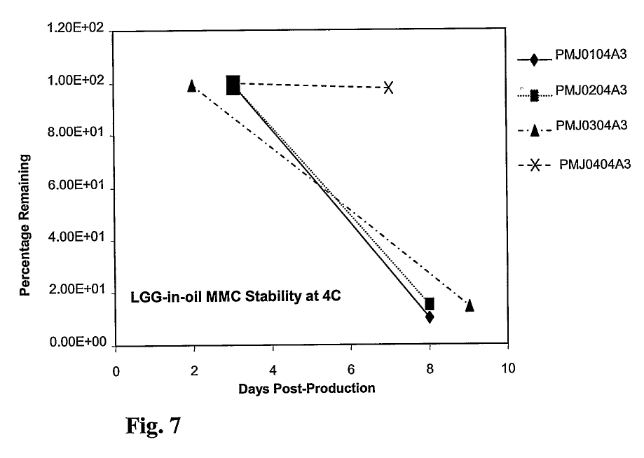

Survival of solid oil microparticulate Lactobacillus GG (LGG) was compared to liquid oil microparticulate LGG over a 30-day storage period at 4 degrees Celsius. Survival of the solid oil microparticulate LGG according to the present invention was significantly higher than the liquid oil microparticulated ones, as shown in FIG. 7.

Heat Exposure Test

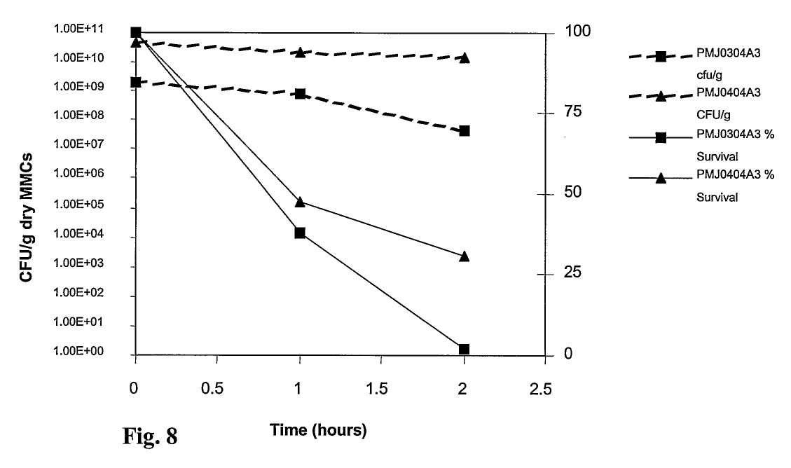

This test provides relative values of viability as colony forming units (colony-forming-units) as a function of a thermal and mechanical stress on the microparticulate probiotic bacteria. Dried microparticles were weighed into sterile BEADBEATER™ tubes containing sterile 2.5 millimeter diameter glass beads (about 10 per tube) and dried in a 50 degrees Celsius oven for 2 hours and viability was assessed. A solution containing 0.9% NaCl, 0.1% peptone, and 50 millimolar EDTA was used to hydrate the microparticles and these were beaten for 3 pulses of 30 seconds. Samples were transferred quantitatively by rinsing with the above solution into a serial dilution series in 0.9% NaCl plus 0.1% peptone. 100 Microliters of sample was plated onto LMRSA plates by spread plating and allowed to absorb right side up for at least 15 minutes. Plates were inverted and incubated at 37 degrees Celsius until counting (usually 3 days later). The solid oil microparticulate probiotic sample exhibited a significantly better survival than liquid oil microparticulate probiotic bacteria, as shown in FIG. 8. The bacteria alone (non-encapsulated) did not survive for 1 hour at this temperature.

### Example 19

Preparation of Gastric Stable Probiotic Microparticle

Preparation of Cocoa Butter—Probiotic Microdroplets without Water Employment

Pure cocoa butter (150 grams) was melted in a microwave and maintained at 36 degrees Celsius. 100 grams dry powder of L. rhamnosus (100 grams LCS-742, Morinaga Milk Industry Co., Tokyo, Japan) and 0.1% w/w magnesium stearate as a lubricator were blended with the molten cocoa butter, using a kitchen blender, while maintaining the temperature at 36 degrees Celsius. The molten cocoa butter/probiotic mixture was atomized using a commercially available fine paint sprayer into a 100 centimeter diameter×200 centimeter height cylinder containing a 5 centimeter layer of dry ice at the bottom. The solid microdroplets in a size range between 10 micrometers and 60 micrometers were harvested after the dry ice sublimed.

Preparation of Gastric Resistant Hydrocolloid Solution

A high amylose starch, lecithin and alginate slurry was prepared as described by Harel (International Patent Application publication no. WO2004/043140).

Preparation of Gastric Resistant Microparticles of the Present Invention

The solidified cocoa butter probiotic microdroplets were blended into 750 milliliters of the gastric resistant hydrocolloid solution using a kitchen blender while maintaining the temperature below the melting point of the cocoa butter. This hydrocolloid slurry was atomized into a calcium chloride bath as described in Example 18. The atomized particles were then freeze-dried and packed under nitrogen in humidity-resistant foil bags.

### Example 20

Preparation of Microparticles Containing Probiotics in Fish Oil-Based Wax Esters

Preparation of Solid Fish Oil

100 Grams of cod liver oil (Twin Lab. Inc., American Fork, Utah, USA) was hydrolyzed with 10 milliliters of methanolic solution containing 12% KOH in a shaker bath at 80 degrees Celsius for 60 minutes, under nitrogen in a tightly closed bottle. The glycerol and catalyst residues were allowed to settle to the bottom and decanted. The hydrolyzed fatty acids were then methylated with 20 milliliters of methanolic solution containing 1% H₂SO₄ at 80 degrees Celsius for 60 minutes under nitrogen in a shaker bath. The methylated fatty acids were allowed to settle on the bottom and the upper phase removed by decanting. The methylated fatty acids were washed first with 5% NaCl and then with deionized water, and dried at 100 degrees Celsius under vacuum in a rotary evaporator. A stoichiometric amount of hexadecanol (0.865 parts of hexadecanol per 1 part of methylated fish oil fatty acids) and 0.6% sodium methoxide were then added. The fatty acids were allowed to esterify with the alcohol under vacuum at 100 degrees Celsius for 3 hours in a rotary evaporator. The alcohol esterified fatty acids (wax esters) were then cooled and washed with water containing 1% H₂SO₄ and then with deionized water. The solid fish oil-based wax ester was dried at 100 degrees Celsius under vacuum and kept at 4 degrees Celsius for later use. The melting point of the solid fish oil-based wax ester was about 34 degrees Celsius.

Preparation of Solid Fish Oil/Probiotic Emulsion

Solid fish oil (100 grams) was melted and maintained at 36 degrees Celsius. An equal amount of dry powder of L. rhamnosus (100 grams LCS-742, Morinaga Milk Industry Co., Tokyo, Japan) was blended with the molten fish oil, using a kitchen blender, while maintaining the temperature at about 36 degrees Celsius.

Preparation of Hydrocolloid Solution

Alginate (1% Prime Algin T-500, Multi-Kern Corp., Raidefield N.J.) was dissolved in distilled water, using a kitchen blender, and maintained at 36 degrees Celsius.

Preparation Microparticles of the Present Invention

The molten fish oil/probiotic paste was blended into 800 milliliters of alginate solution using a kitchen blender while maintaining the temperature above the melting point of the solid fish oil (34 degrees Celsius). The slurry was then internally cross-linked by rapid mixing with 1 normal monobasic calcium phosphate and pouring the mixture into a setting mold. The internally set material from the setting mold was chopped then freeze-dried. The resulting particles can be used “as-is” or milled and sifted to a specific size range between 10 and 5,000 micrometers. The dry powdered microparticles were packed under nitrogen in humidity resistant foil bags. Both approaches have been tested.

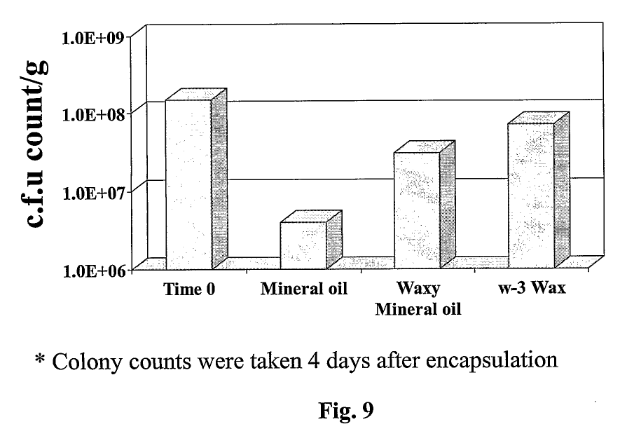

The solid fish oil-based microparticles retained similar advantages of the cocoa butter based microparticles with the additional advantage of being a superior water barrier, due to the waxy fish oil, which protected the probiotics in open air and humid storage conditions, as shown in FIG. 9.

### Example 21

Enzyme-Containing Microparticles

Hydrocolloid slurry was prepared according to Example 18 except for the addition of 100 grams of SAVINASE® (Novozymes, Denmark) and use of a mixture of equal amounts of natural beeswax and mineral oil (50 grams each) as the solid oil with a melting point of 41 degrees Celsius. The solid oil-enzyme mixture was added to a 1% gelatin hydrocolloid solution while maintaining the temperature above the melting point of the solid oil (45 degrees Celsius). The slurry was then atomized through a nozzle into icy water bath containing 1 molar HCl. The microparticles were harvested on a fine mesh screen (68 micrometers) and gently rinsed with 1% citric acid. The wet microparticles were vacuum dried and packed under nitrogen in humidity-resistant foil bags.

For determination of loading and encapsulation efficiencies of the microparticles: Microparticles were accurately weighed (<100 milligrams) in a microcentrifuge tube. 200 Microliters of dimethyl sulfoxide (DMSO) was added. The particle matrix was dissolved by vortexing. To this sample, 0.8 milliliters of a solution containing 0.05 normal NaOH, 0.5% SDS and 0.075 molar Citric acid (trisodium salt) was added. The tubes were sonicated for 10 minutes at 45 degrees Celsius, followed by a brief centrifugation at 5,000 rpm for 10 minutes. Aliquots of the clear DMSO/NaOH/SDS/citrate solution were taken into wells of a microplate and analyzed for protein content using the Bradford assay method. The encapsulation efficiency of the enzyme in the solid oil microparticle composition of the present invention was significantly higher than microparticles with no solid oil (Table 4).

TABLE 4
Retention of SAVINASE ® in solid or
liquid oil containing microparticles

| Oil state | % Retention of SAVINASE ® |
|---|---|
| Liquid oil microparticles | 20% |
| Solid oil microparticles | 85% |

% Retention of SAVINASE ® was determined by measuring the protein content before and after atomizing of the slurry.

### Example 22

Microparticles Containing Antibiotics Against Common Pathogens

Alcohol esters of polyunsaturated fatty acids obtained from a DHA-rich algal oil (Martek Biosciences Corp., Columbia Md.) are prepared according to Example 20 to produce a solid DHA algal oil. 100 Parts per million tetracycline is added to 100 grams of the solid DHA algal oil blend according to Example 18 and sprayed in a cylindrical column containing dry ice as described in Example 19. The solidified microdroplets containing tetracycline are then harvested and added to a mixture of 1% chitosan and 0.5% carboxymethyl cellulose hydrocolloid solution. The slurry is then atomized through a nozzle into a water bath containing 4% tripolyphosphate. The microparticles are harvested on a fine mesh screen (68 micrometers) and gently rinsed with cold water. The wet microparticles are then vacuum dried and packed under nitrogen in humidity resistant foil bags.

The following assay is used to determine the efficacy of the tetracycline microparticles against common bacteria. 20 milligrams of dry microparticles is dissolved in 100 microliters of DMSO. The solution is then added to Mueller Hinton broth and the solution is diluted to 50 microliters volumes, with a test compound concentration of 0.1 microgram per milliliter. Optical density (OD) determinations are made from fresh log-phase broth cultures of the test strains. Dilutions are made to achieve a final cell density of 10⁶ colony-forming-units per milliliters. At OD=1, cell densities for different genera should be approximately: for Escherichia coli, 10⁹ colony-forming-units per milliliters; for Staphylococcus aureus, 10⁸ colony-forming-units per milliliters; and for Enterococcus sp., 10⁹ colony-forming-units per milliliters.

50 Microliters of the cell suspensions are added to each well of microplates. The final cell density should be approximately 5×10⁵ colony-forming-units per milliliters. These plates are incubated at 35 degrees Celsius for approximately 18 hours. The plates are read with a microplate reader and are visually inspected when necessary. The Minimum Inhibitory Concentration (MIC) is defined as the lowest concentration of the tetracycline compound that inhibits growth.

### Example 23

Microparticles Containing Carotenoids and Having Bioadhesive and Permeability Enhancing Properties

A bioadhesive polymer and/or permeability enhancing material may be included in the microparticle to increase the contact time between the bioactive agent and the mucosal membranes in the gastrointestinal tract and to improve uptake.

In the present example a combination of chitosan as a bioadhesive polymer and alcohol esters of highly unsaturated fatty acids as permeability enhancers are used.

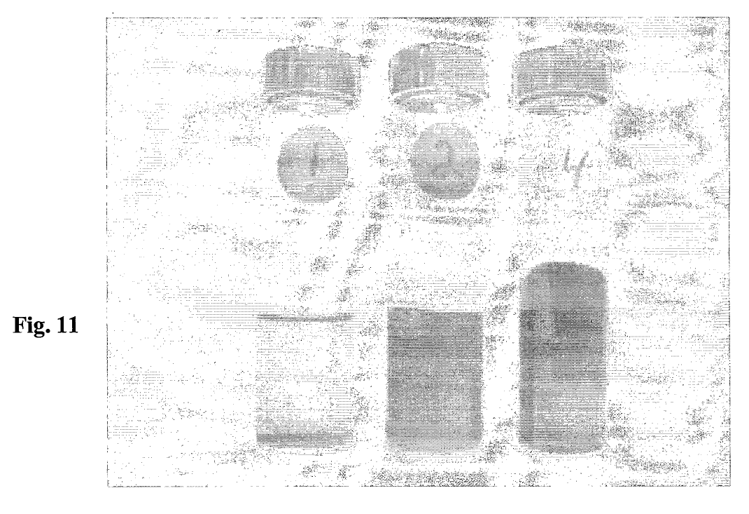

Solid waxy alcohol esters of highly unsaturated fatty acids are prepared according to example 20 except that algal DHA (docosahexaenoic acid) oil (Martek, Columbia Md.) is used instead of fish oil and Lucantin Pink 20% (BASF, Limburgerhof, Germany) used as the bioactive agent. The O/W emulsion containing solidified waxy droplets is then combined with a bioadhesive mixture of 1% chitosan and 0.5% carboxymethyl cellulose hydrocolloid solution. The slurry then atomized through a nozzle into a water bath containing 4% tripolyphosphate. The microparticles are harvested on a fine mesh screen (68 micrometers) and gently rinsed with cold water. The wet microparticles are vacuum dried and packed under nitrogen in humidity resistant foil bags. The resulting microparticles are water insoluble and retained the Lucantin Pink pigment in both water and gastric juice as shown in FIG. 11. The pigment is completely released from the particles after exposure to intestinal juice.

These microbeads are bioadhesive due to the presence of the chitosan and carboxymethyl cellulose polymers, while the waxy DHA oil provides both a humidity and oxygen barrier and enhances membrane permeability. Overall, these microparticles improve the bioavailability and uptake of astaxanthin to the animal.

### Example 24

Microparticles for Treatment of Gastrointestinal Ulcer

Nizatidine is a known pharmaceutical agent that is used in the treatment of gastrointestinal ulcer. Its chemical name is N-[2-[[[2-[(dimethylamino)methyl]-4-thiazolyl]methyl]thio]ethyl]-N′-methyl-1-2-nitro-1,1-ethenediamine. U.S. Pat. Nos. 4,375,547 and 4,382,090, herein incorporated by reference, describe how to produce Nizatidine.

In the present invention, nizatidine or modified nizatidine is mixed with cocoa butter according to Example 18. The molten mixture and a warm hydrocolloid solution (36 degrees Celsius) containing 0.2% alginate 5% sodium carbonate and 10% dibasic calcium phosphate are delivered into an ultrasonic atomizer, through separate inlets. The nizatidine solution flows at 1 milliliter per minute and the hydrocolloid solution flows at 1.5 milliliters per minute. Upon the onset of ultrasonic vibration of the atomizer, both liquids are fragmented into microdroplets. The microdroplets are then harvested in ice chilled water bath containing 1% acetic acid. The microparticles are harvested on a fine mesh screen (68 micrometers) and gently rinsed with cold water. The wet microparticles are freeze-dried and may also be lubricated at this point with magnesium stearate. These microbeads will gradually release their contents to gastric environment because the entrapped sodium carbonate will be converted to CO₂ gas at the low pH of the stomach. This will cause the microbeads to float on the surface of the gastric juices while gradually releasing their contents through the porous matrix of the alginate, providing instant and long lasting relief.

### Example 25

Microparticles for Treatment of Diabetes

Human insulin is a known pharmaceutical agent that is used in the treatment of diabetes. Commercially available insulin is not extracted from the human pancreas, but can be prepared biosynthetically from cultures of genetically modified Escherichia coli or Saccharomyces cerevisiae. Human insulin is the subject of U.S. Pat. Nos. 5,474,978 and 5,514,646, herein incorporated by reference, which describes the preparation of that drug.

Glucagon is also a pharmaceutical agent used in the treatment of diabetes. This is a naturally occurring polypeptide that can either be isolated or synthesized.

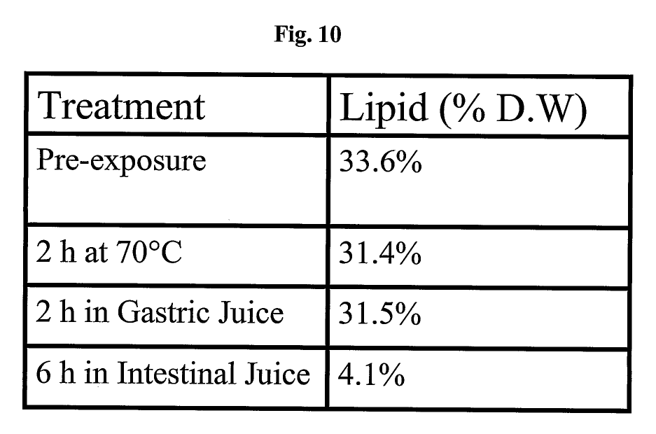

In the present invention, insulin and glucagon (10% and 40%, respectively) are both mixed with 50% molten hydrogenated vegetable oil and spray chilled to form solid microdroplets as in Example 19. A hydrocolloid slurry (900 milliliters) containing 2% high shear modified high amylose starch and 1% alginate prepared in deionized water is brought to room temperature and mixed with the insulin/glucagon droplets. The final slurry then atomized into ice chilled water bath containing 2% calcium chloride. The microparticles are harvested on a fine mesh screen (68 micrometers) and gently rinsed with cold water. The wet microparticles are freeze-dried and may also be lubricated at this point with magnesium stearate. These microbeads will resist gastric degradation because of the presence of non-digestible starch in the alginate matrix, while gradually releasing the content to the intestinal environment because the high pH and phosphate rich environment of the intestine triggers release of the cross-linked alginate. These properties are exemplified in FIG. 10, which demonstrates a gastric retention of the oil droplets within the microparticles matrix as apposed to their substantial release in assimilated intestinal fluids.

### Example 26

Microbeads for Enhancing the Animal Immune System

Thymosin alpha is a known pharmaceutical agent that is generally used to enhance the animal immune system and in the treatment of hepatitis B in human. The sequence and synthesis of human thymosin alpha is described in U.S. Pat. No. 4,079,127, and is herein incorporated by reference.

Microbeads comprising thymosin alpha and deoxycholate (as a permeability enhancer) are formulated according to Examples 19 and 23. The thymosin alpha, molten fish oil wax ester mix, and the warm chitosan hydrocolloid solution are delivered into a coaxial ultrasonic atomizer as described in U.S. Pat. No. 6,767,637 using syringe pumps at controlled flow rates. The thymosin solution flows through the inner nozzle at 0.5 milliliter per minute and the hydrocolloid solution flows through the outer nozzle at 2 milliliters per minute. The ultrasonic vibration of the atomizer causes both liquids to fragment and coalesce into microdroplets in the air. The microcapsules are cross-linked by spraying into a capture tank of ice-chilled water containing 4% tripolyphosphate. The microparticles are harvested on a fine mesh screen (68 micrometers) and gently rinsed with cold water. The wet microparticles are vacuum dried or freeze-dried. These microbeads will protect and immobilize the Thymosin peptide from humid and oxidative environment and from gastric degradation, while providing bioadhesive and penetration enhancing properties for the drug.

### Example 27

A Yogurt Food Product Containing Both Probiotic and DHA Oil Microparticles

Gastric protected microparticles containing Lactobacillus acidophilus and solid algal DHA oil (modified from DHASCO, Martek, Columbia Md. according to Example 22) are prepared according to Example 19. A yogurt composition is then prepared by mixing 100 grams of DANNON® brand plain, low fat yogurt with 2.5 grams of the above wet microbeads. The final food product contains probiotic counts of approximately 5×10⁶ colony-forming-units per gram and 400 milligrams of DHA per 100 grams yogurt.

### Example 28

A Chocolate Bar Food Product Containing Probiotic Microparticles

Gastric protected microparticles containing L. rhamnosus are prepared according to Example 29 using cocoa butter as the source of solid fat. A chocolate bar is prepared by mixing milk chocolate composition using the formulation in Table 5.

TABLE 5

| | |
|---|---|
| Sucrose | 50% |
| Cocoa Butter | 20.5% |
| Whole Milk Powder | 18% |
| Chocolate Liquor | 11% |
| Lecithin | 0.5% |
| Vanillin | 0.01% |

The milk chocolate mixture is mixed for 30 minutes at 45 degrees Celsius. Then the chocolate mix is cooled to 36 degrees Celsius and the probiotic microparticles added and the temperature further reduced to 28 degrees Celsius with aggressive shear to produce stable cocoa butter crystals, which are then molded to a final bar and further cooled to room temperature. The final food product contained probiotic count of 5×10⁷ colony-forming-units per gram of chocolate.

### Example 29

An Infant Formula Containing Microparticles

Microparticles containing Lactobacillus GG (Valio Corp, Finland) are prepared according to Example 18 followed by a sieving into 2 size groups of microbeads (above 50 micrometers and below 50 micrometers). An infant formula is prepared by mixing 99 grams of NUTRAMIGEN® (Mead Johnson) with 1 gram of the small size microparticles (below 50 micrometers). The final product contains about 10⁸ colony-forming-units of Lactobacillus GG per 100 grams infant formula.

### Example 30

An Infant Formula Containing DHA and ARA Oil Microparticles

DHA and ARA oil-based microparticles (DHA and ARA oils from DHASCO and ARASCO, Martek, Columbia Md.) are prepared according to Examples 18 and 19 followed by a sieving into two size groups of microbeads (above 50 micrometers and below 50 micrometers). The DHA and ARA oil (Martek Biosciences Corp., Columbia, Md.) are mixed in a proportion of 10% DHA oil and 20% ARA oil with 20% dibasic calcium phosphate, 20% starch and then with 30% molten cocoa butter. The molten mixture is sprayed chilled as in Example 18 and the solidified microdroplets are collected. The DHA/ARA/cocoa butter droplets are then added to alginate hydrocolloid solution and microparticulate as described in Example 19. An infant formula is prepared by mixing 99 grams of Enfamil® (Mead Johnson, Evansville, Ill.) with 1 gram of the small size microparticles (below 50 micrometers). The final product contains 400 milligrams ARA and 200 milligrams of DHA per 100 grams infant formula.

### Example 31

Microbeads Feed for Fish and Crustacean Larvae

A mixture of 50% waxy fish oil, 20% Lactobacillus rhamnosus, 20% algal biomass (e.g., Nannochloropsis sp.) and 10% fishmeal (on a dry weight basis) is prepared and added to the alginate hydrocolloid solution described in Example 18. The slurry then atomized through a nozzle into a water bath containing 2% calcium acetate. The microbeads at a size distribution between 50-200 micrometers are harvested and gently rinsed with fresh water. The wet microparticles are then vacuum dried or stored wet under vacuum in 4 degrees Celsius for delivery to fish or shrimp larvae.

### Example 32

Microbeads Containing Carotenoids for Coloring Salmon and Trout Fish

Forty grams of Natural astaxanthin (NATUROSE™, Cyanotech Corporation Kailua-Kona, Hi.) is mixed vigorously for 1 hour into a molten mix of 50 grams cocoa butter and 10 grams lecithin. This mixture is then emulsified by adding 100 grams of water with continued vigorous mixing. The mixture is then chilled to solidify the microdroplets. The astaxanthin-containing solidified microdroplets are then added to alginate hydrocolloid slurry at room temperature as in Example 18. The mixture is atomized and the microbeads harvested and vacuum dried. The astaxanthin-containing microbeads can then be blended with a standard feed formulations for fish, or other animals (e.g., chickens) at a level of about 40 milligrams astaxanthin per kilogram feed, and can be fed to promote the coloring of the flesh or eggs.

### Example 33

Feeding Cats and Dogs with Extruded Feeds Containing Probiotic Microparticles

A standard commercial dog food is amended with 1% of microbead preparation from Example 19. The standard dog chow can be mixed with the microbeads prior to pelleting or cold extrusion, or can be top-coated with oil containing the microbead preparation. The resulting feed can be fed to pets for induction of healthy microflora.

### Example 34

Production of Bifidobacterium-Containing Infant Formula

Pure cocoa butter (15 kilograms) is melted and maintained at 36 degrees Celsius in stirred, temperature controlled storage container. 10 Kilograms dry powder of Bifidobacterium (e.g., BB12, Nestle, Switzerland) is maintained in a dry form in a second temperature controlled storage container (maintained at 10 degrees Celsius). Gastric-resistant hydrocolloid (100 kilograms) comprising 2% high amylose starch (HYLON™ VII, National Starch and Chemical, Bridgewater, N.J.) and 1% alginate (Prime Algin T-500, Multi-Kern Corp., Raidefield N.J.) is maintained at 36 degrees Celsius in a third temperature controlled storage container. The probiotic sample is transferred to the melted cocoa butter and vigorously agitated for 1 minute. The Cocoa butter/probiotic mixture and the gastric resistant hydrocolloid mixture are then pumped simultaneously into an in-line mixer/emulsifier (1 part cocoa butter/probiotic mixture to 4 parts gastric-resistant hydrocolloid mixture) and the single outlet stream flows into an atomization nozzle at a rate of 1 kilogram per minute. The microparticles are captured in a chilled tank (10 degrees Celsius) containing 1% calcium chloride and continuously harvested and rinsed with fresh cold water so that the maximum contact time of the bacteria with the calcium chloride bath is no more than 15 minutes. Following washing, the microparticles are air dried with forced cold air for 15 minutes before freezing and further drying under vacuum.

Dried microparticles have a composition that is approximately 53% cocoa butter, 36% Bifidobacteria, 7% high amylose starch, and 3% alginate and the live bacterial count should be in the order of 10¹⁰ per gram. An infant formula (e.g., Enfamil®, Mead Johnson Corp, Evansville, Ind.) is then amended by dry mixing 1.0 gram of the final microparticle material with 100 grams of infant formula to provide a final live bacterial count of 10⁸ colony-forming-units per 100 grams of formula. The mixed formula is then vacuum packed and is ready for consumption. Because this microparticle formulation results in significant gastric protection of the probiotic bacteria, a low-dose formula is also prepared by adding 1.0 gram of the final microparticle material to 10 kilograms of infant formula, resulting in a final live bacterial count of 10⁶ colony-forming-units per 100 grams of formula.

The disclosure of every patent, patent application, and publication cited herein is hereby incorporated herein by reference in its entirety.

While this invention has been disclosed with reference to specific embodiments, it is apparent that other embodiments and variations of this invention can be devised by others skilled in the art without departing from the true spirit and scope of the invention. The appended claims include all such embodiments and equivalent variations.

1-78. (canceled)

79. A particle suitable for oral administration of a vaccine to an animal comprising multiple compartments;
wherein the particle comprises the vaccine, an indigestible polymer, a lipid, and an emulsifier;
wherein the lipid is dissoluble in the animal and more dissoluble in one compartment of the animal than in another compartment of the animal;
wherein the lipid and the vaccine are suspended in a matrix comprising the indigestible polymer;
wherein the matrix and the lipid are emulsified, and the vaccine is suspended in the form of an emulsion;
wherein the indigestible polymer comprises chitosan and inulin; and
wherein the particle comprises on a dry particle weight basis:
(i) not more than 50% of lipid;
(ii) at least 5 percent of the polymer matrix;
(iii) up to 70 percent of the vaccine; and
(iv) 0.1-10% of the emulsifier.

80. The particle of claim 79, wherein the polymer matrix is cross-linked.

81. The particle of claim 79, wherein the polymer matrix further comprises a protein.

82. The particle of claim 81, wherein the protein is selected from the group consisting of gelatins, albumins, glutens, whey proteins, caseins, zeins, and any combination thereof.

83. The particle of claim 79, wherein the polymer matrix makes up not less than 10% of the dry weight of the particle.

84. The particle of claim 83, wherein the polymer matrix makes up not more than 25% of the dry weight of the particle.

85. The particle of claim 84, wherein the polymer matrix makes up not more than 75% of the dry weight of the particle.

86. The particle of claim 79, wherein the animal is an aquatic animal.

87. The particle of claim 86, wherein the animal is selected from the group consisting of fish, mollusks, rotifers, and crustaceans.

88. The particle of claim 79, wherein the animal is a farm animal.

89. The particle of claim 79, wherein the animal is a winged animal.

90. The particle of claim 79, wherein the lipid is a vegetable oil.

91. The particle of claim 90, wherein the vegetable oil is selected from the group consisting of safflower oil, sunflower oil, canola oil, corn oil, peanut oil, pine oil, lilac oil, and any combination thereof.

92. The particle of claim 79, wherein the emulsifier is selected from the group consisting of monoglycerides, sorbitan esters, propylene glycol esters, lecithins, polysorbates, sucrose esters of fatty acids, and any combination thereof.
